(19) United States
(12) Patent Application Publication
(10) Pub. No.: US 2025/0273347 A1
(43) Pub. Date: Aug. 28, 2025

(54) AI-ASSISTED UNIT OF MEASURE STANDARDIZATION WITH CONTEXT AND STANDARDS

(71) Applicant: Jacob Barhak, Cedar Creek, TX (US)

(72) Inventor: Jacob Barhak, Cedar Creek, TX (US)

(21) Appl. No.: 19/209,218

(22) Filed: May 15, 2025

Related U.S. Application Data
(63) Continuation-in-part of application No. 17/176,152, filed on Feb. 15, 2021.

(51) Int. Cl. G16H 50/50

(52) U.S. Cl. CPC G16H 50/50

(57) ABSTRACT

A system for standardizing units of measure, comprising: a database comprising standardized units, unit text variations, unit context information, and interpreter standards; a neural network configured to process at least one of: unit text input, unit context input, and interpreter input to generate suggested standardized unit mappings; a nearest unit search component configured to match neural network outputs to permitted standardized units from the database; and an output component configured to provide a standardized unit based on the matched neural network outputs.

### CROSS-REFERENCE TO RELATED APPLICATIONS

This application is a continuation-in-part of U.S. patent application Ser. No. 17/176,152 filed on Feb. 15, 2021 which claims priority to and is a continuation-in-part of U.S. patent application Ser. No. 15/466,535 entitled “Analysis and Verification of Models Derived From Clinical Trials Data Extracted From a Database” filed on Mar. 22, 2017, which claims priority to U.S. Provisional Patent Application No. 62/315,578 entitled “Reference Model for Disease Progression Using Object Oriented Population Generation” filed on Mar. 30, 2016, and to U.S. Provisional Patent Application No. 62/326,052 entitled “Reference Model for Disease Progression Using Model Combination” filed on Apr. 22, 2016.

### BACKGROUND

In some particular situations, data related to clinical studies can be stored in a database. Clinical studies are performed by scientists on a population of subjects often to study an aspect of health. In various situations, a clinical study can examine how behaviors, diet, medications, and the like can influence an aspect of human health. The clinical studies document characteristics of the population participating in the clinical studies. The clinical studies can also indicate the effect that particular behaviors, diet, and/or medications have on the populations that are the subjects of the clinical studies. Additionally, the clinical studies can provide models based on the data obtained from the clinical studies. where the models can indicate the amount of influence that a particular variable has on one or more aspects of the health of individuals. The models can also indicate the progression of a disease in individuals and provide information about the transitions between one state of a disease to another. The models derived from clinical studies often indicate assumptions made by the scientists conducting the research about the progression of a disease.

Clinical studies can provide useful information to the public about behaviors, diet, and/or medications that can influence the health of individuals. In addition, access to clinical study data can be used to test the efficacy of the models derived from the clinical study data. The amount of clinical study data available to the public has been on the increase. In a particular example, the website clinicaltrials.gov provided by the United States National Institutes of Health provides a repository for storing clinical studies data that is accessible to the public. However, the extraction and manipulation of data from databases storing clinical study data can present challenges.

### DESCRIPTION OF THE DRAWINGS

The Detailed Description is set forth with reference to the accompanying figures. In the figures, the left-most digit(s) of a reference number identifies the figure in which the reference number first appears. The use of the same reference numbers in different figures indicates similar or identical items.

FIG. 1 shows a schematic diagram of an example framework to determine the fitness of clinical study models to predict the progression of a biological condition.

FIG. 2 shows a schematic diagram of a framework for extracting information from clinical studies data to generate populations used to evaluate models that predict the progression of a biological condition.

FIG. 3 shows a schematic diagram of a framework showing the use of object oriented techniques to generate virtual populations used to verify models derived from clinical data.

FIG. 4 shows a schematic diagram of a framework to determine a combination of models that predicts progression of a biological condition.

FIG. 5A and FIG. 5B show examples of using gradient descent techniques to determine a minimum for an aggregate fitness function that identifies the contributions of each individual model to the aggregate fitness function.

FIG. 6 shows a block diagram of an example computing device to evaluate models derived from clinical data using a cooperative framework with some competitive elements.

FIG. 7 is a flow diagram of an example process to evaluate models derived from clinical data using a cooperative framework with some competitive elements.

FIG. 8 is a block diagram of a framework to incorporate user input into the process of generating aggregate models to predict the progression of a biological condition.

FIG. 9 is a flow diagram of an example process to incorporate user input into generating an aggregate model to predict the progression of a biological condition.

FIG. 10 is a block diagram showing the progression of disease states of COVID19 and models used to determine progression from one state to another.

FIG. 11 is a diagram including example user interfaces that show results of combinations of models and fitness scores for iterations of the techniques and frameworks described herein.

FIG. 12 shows a schematic diagram of an exemplary AI-based unit mapping system.

FIG. 13 illustrates a schematic diagram of an exemplary unit conversion system that builds upon the unit mapping system.

FIG. 14 illustrates a schematic diagram of an exemplary system employing multiple unit mapping models with a reasoning model.

FIG. 15 illustrates a schematic diagram of an exemplary system employing multiple conversion systems with a reasoning model.

FIG. 16 illustrates a user interface for the AI-assisted unit standardization system.

### DETAILED DESCRIPTION

This disclosure is directed to the analysis and verification of models derived from data extracted from a database. In particular, this disclosure describes implementations that extract clinical study data from a database and analyze models derived from clinical studies data. The analysis of the models can be used to verify the results of the clinical studies from which the models were derived. Additionally, the analysis of the models can identify a combination of models that can be used to predict health outcomes of one or more biological conditions for one or more populations.

In particular, the implementations described herein include extracting data related to clinical studies from a database storing clinical study data. In some cases, the data extracted from the database can correspond to clinical studies that were conducted with respect to one or more biological conditions. Additionally, the data extracted from the database can correspond to one or more populations. Clinical study data can be extracted from a database based on a query. In some cases, the query can include a text query that includes keywords that are used to identify clinical studies corresponding to the keywords. In particular implementations, specific instructions can be accessed during the extraction of information from a clinical studies database to extract particular information from the clinical studies database. For example, instructions can be accessed during the extraction of clinical studies data to specifically obtain population data from clinical studies that correspond with a query. To illustrate, a query can be provided that is related to obtaining data from clinical studies where diabetes was studied and instructions can be utilized to extract characteristics of the populations of those clinical studies, such as age, weight, biological indicators (e.g., cholesterol levels, high density lipoprotein (HDL) levels, etc.). The use of particular sets of instructions to extract data from a clinical studies database can reduce the computing resources used to obtain specific information from the clinical studies database. In some implementations, the extraction of clinical study data from one or more databases can take place in multiple phases. In particular implementations, a first phase can include extracting information related to a number of clinical studies from a database, while a second phase can include filtering the extracted information based on a particular filtering criteria.

Observed data obtained from clinical studies, can be used to evaluate the various models derived from multiple other datasets. A model can be evaluated using a number of populations that can have at least some characteristics that are different from the population that participated in the clinical study that was used to derive the model. The results from the evaluation can be compared against observed outcomes from the same clinical study or from different clinical studies to determine a fitness of the model for predicting outcomes for a biological condition associated with the model. In previous situations, a competitive framework was utilized to compare the fitness of different models based on evaluating the models with a set of populations. However, the competitive framework utilized large numbers of memory and processing resources that continued to increase as the number of models being evaluated increased. In particular, the amount of computing resources and memory resources utilized to evaluate models derived from clinical study data increases close to exponentially as the number of models being evaluated increases.

In contrast to previous scenarios, the implementations described herein utilize a cooperative framework in conjunction with some competitive elements in the evaluation of models described from clinical study data. In particular, a linear combination of models can be evaluated with the contribution of each of the models being indicated by a coefficient associated with the model. The minimum for the linear combination of models can be determined in order to evaluate the coefficients for each model that provide the best fitness for predicting the progression of a biological condition. The coefficients that have the greatest contribution to the linear combination can be identified as the models that have the best fitness for predicting the progression of a biological condition. In some particular implementations, gradient descent techniques can be utilized to evaluate the linear combination of models. By utilizing a cooperative framework with some competitive elements to evaluate the fitness of models derived from clinical studies data rather than a competitive framework, the number of processing and memory resources increases at merely a linear rate per iteration when the number of models being evaluated increases as opposed to an almost exponential rate.

Additionally, a cooperative framework with some competitive elements can identify information about models derived from clinical data that a competitive framework is unable to identify. For example, a cooperative framework with some competitive elements can determine a combination of models that can effectively predict the progression of a biological condition and the contributions of each model to the combination. Conversely, a framework that is simply competitive can merely be used to identify the performance of a single model with respect to other individual models, but does not provide any indication as to how the models that predict the same phenomenon can be combined to provide a composite model to predict the progression of a biological condition nor can the competitive model that is discrete by choice of model be as accurate as a cooperative model that merges models continuously.

The evaluation of models derived from clinical study data for the purposes of predicting disease progression can be performed by generating a number of populations from the clinical study data and evaluating various models in light of characteristics of the different populations. In some cases, certain models may have a higher fitness than other models with respect to different populations. To generate the populations used to evaluate models derived from clinical study summary data, characteristics of various populations can be analyzed and virtual populations can be generated from the actual populations that participated in the clinical studies. Access to personalized clinical study data is restricted, yet summary data is available publicly and unrestricted. Therefore, generating a synthetic population increases the amount of information available to model. In this way, the aggregate population from a number of different clinical studies can be utilized to determine a number of virtual populations that can be used to evaluate models that predict the progression of a biological condition, where the virtual populations can have different characteristics from the clinical study populations. For example, a virtual population used to evaluate models predicting the progression of diabetes can have blood pressure, age, triglyceride, HDL, and low density lipoprotein (LDL) distributions that are derived from a number of clinical study populations, but do not actually match the populations that participated in the clinical studies, although describing similar statistics.

In generating the virtual populations used to evaluate models that predict the progression of a biological condition, object oriented techniques can be implemented. For example, objects can be created that include characteristics of one or more populations that participated in one or more clinical studies. To illustrate, an object can be created that includes rules that generate distributions for age, gender, height, and weight for a population that participated in a clinical study that is considered a default for a population. In another example, an object can be created for another population that indicates an objective from the clinical data associated with the population. In this way, a virtual population can be generated using the characteristics of one clinical study population and an objective of another clinical study population by creating an object for the virtual population that inherits the population characteristic generating rules from the first population that is considered as representing the default population structure and the objective from the second population representing specific summary statistics found in a certain trial.

By allowing populations to be generated using object oriented techniques, the implementations described herein enable flexibility in the characteristic generating rules and objectives utilized to generate virtual populations and also result in reducing the amount of computing resources utilized to generate a population. In particular, rather than recreating the characteristics and/or objectives of each clinical study population utilized to generate a new, virtual population, the objects associated with the clinical study populations can simply be inherited by the object of the new, virtual population. Furthermore, characteristics that may be missing from a particular population can be filled in by inheriting the missing characteristics from another population. This adds to the flexibility of the implementations described herein with respect to conventional techniques that are limited in the way that population characteristics can be combined to generate a virtual population used to evaluate the fitness of models that predict the progression of biological conditions.

Furthermore, the simulations that are performed with respect to the evaluations of the aggregate models can be performed concurrently and using parallel computing techniques. The concurrent processing of simulation and the using multiple processors in parallel reduces the amount of time needed to evaluate the aggregate models.

In addition, the disclosure is directed to incorporating user input into generating aggregate models to estimate progression of biological conditions. In existing scenarios, results of clinical studies can be identified using one or more definitions that correspond to the outcomes of an individual in which a biological condition is present. In various examples, definitions can change over time. For example, a given source of definitions of outcomes of biological conditions can change over time. In one or more illustrative examples, the International Statistical Classifications of Diseases (ICD) can include definitions and codes for various outcomes of biological conditions. These definitions and codes can be modified in different versions of the ICD.

In these situations, results from clinical studies recorded during one period of time can be represented with one or more definitions that are different than results from clinical studies recorded during another period of time even though the results may be considered to be the same or similar. For example, a clinician may consider outcomes for individuals participating in clinical studies at different times to be the same or similar, but the results of the clinical studies can be recorded using different definitions for the outcomes based on differing versions of the definitions. In additional examples, the results of clinical studies can be recorded using different definition systems or classifications. To illustrate, results from a first set of clinical studies can be recorded using a different outcome classification system than a second set of clinical studies.

The reporting of results of clinical studies using different classification or definition systems can cause models that predict outcomes of biological conditions using clinical studies data to have decreased accuracy because the results of the clinical studies are not recorded consistently. The techniques and systems described herein incorporate user input into the validation of models that predict outcomes of biological conditions for populations of individuals. The user input can increase the accuracy of the models by harmonizing results of clinical studies that may be recorded using different systems of definitions or classifications. The user input can correspond to input from experts in a given field that indicates a measure of accuracy of the results of one or more clinical studies that are being used to generate models that predict the outcome of biological conditions.

Conventional techniques that may be used to incorporate user input into generating models that predict the outcome of biological conditions can lead to an increase in the amount of computing resources used to train and validate the models. In particular, conventional techniques and systems incorporate the user input into the simulations used to generate an aggregate model to predict outcomes of biological conditions. In these scenarios, the simulations for each population and for each model combination would be performed for the input obtained from each expert. Thus, the computational resources utilized to incorporate each expert's input would be similar to the computational resources utilized to incorporate each model into the simulations.

As a result, the computational resources utilized by conventional systems and techniques can add hours, if not days, to the computational time used to generate an aggregate model with user input depending on the number of experts providing input and the number of processing cores used to generate the aggregate model. However, the techniques and systems described herein result in a minimal increase of computational resources utilized to generate an aggregate model by decoupling the simulation phase of model generation from the validation phase and adding the expert input into the validation phase. In this way, the computational resources utilized to add the input from 100 experts is similar to the computational resources utilized to add the input from 10 experts. Accordingly, the techniques described herein improve the functioning of systems that generate models to predict the outcome of biological conditions by reducing the amount of computing resources used to generate the models when compared with conventional techniques.

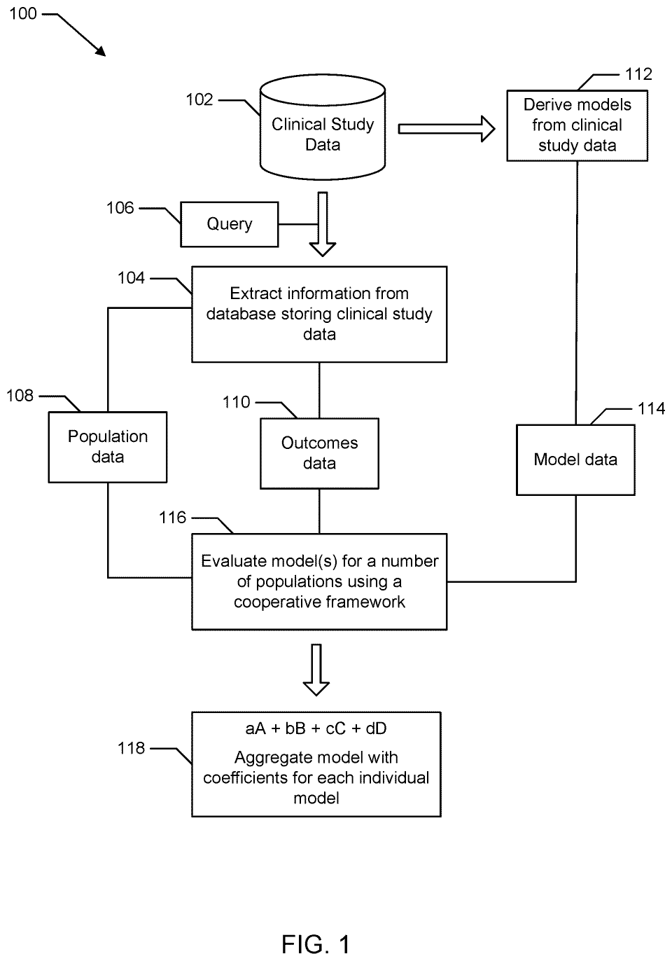

FIG. 1 is a schematic diagram of an example framework 100 to determine the fitness of clinical study models to predict the progression of a biological condition. The framework 100 includes clinical study data 102. The clinical study data 102 can be stored in one or more databases. The clinical study data 102 can be accessible by computing devices via an interface. In some cases, the interface can include a webpage that enables access to the clinical study data 102 being stored by the one or more databases. In other implementations, the clinical study data 102 can be accessed via a computing device application. In particular, the clinical study data 102 can be accessed using an app executing on a mobile computing device, such as a tablet computing device or a smartphone.

The clinical study data 102 can include information related to clinical studies that have been conducted by scientists and/or scientific organizations. The clinical studies can be related to various biological conditions. In some scenarios, the biological conditions can include diseases. In particular implementations, the biological conditions can be related to a level of an analyte present in subjects of the clinical studies. In some situations, the clinical studies can examine the effects of one or more factors on a biological condition. The factors can include characteristics of subjects participating in the clinical studies, such as age, weight, gender. The factors that can affect a biological condition can also include levels of analytes measured in subjects. For example, factors that can affect a biological condition can include cholesterol levels, triglyceride levels, HDL levels, LDL levels, and the like. Additionally, the factors that can affect a biological condition can include behaviors of subjects participating in clinical studies. To illustrate, the factors can include information related to diet (e.g., servings of fruits and/or vegetables per day), exercise, sleep, and so forth.

The framework 100 includes, at 104, extracting information from a database storing the clinical study data 102. The information can be obtained through a query 106. The query 106 can include one or more keywords that can form the basis of a search of the clinical study data 102. In some cases, the query 106 can include keywords directed to a particular biological condition. In additional situations, the query 106 can include keywords related to characteristics of populations participating in clinical studies. The query 106 can also include keywords corresponding to factors that can affect the progression of a biological condition. In an illustrative example, the query 106 can include keywords corresponding to diabetes, heart attack, and/or stroke. In this situation, clinical studies that include the keywords diabetes, heart attack, and/or stroke will be identified in the clinical study data 102.

The extraction of information from the clinical study data 102, at 104, can include parsing one or more databases that store the clinical study data 102 for clinical studies that include one or more keywords of the query 106. Additionally, after identifying clinical studies that correspond to the query 106, particular information can be extracted from the clinical study data 102. For example, instructions can be involved in the extraction of information from the clinical studies data 102 that cause certain portions of information included in individual clinical studies to be extracted, while leaving behind other portions of information included in the individual clinical studies.

In the illustrative example of FIG. 1, the information extracted from the clinical studies data 102 can include population data 108 and outcomes data 110. The population data 108 can include information related to the populations that participated in the individual clinical studies that provided the clinical study data 102 including baseline population distributions. The outcomes data 110 includes results from the clinical studies. In some examples, the outcomes data 110 can include information indicating a progression of a biological condition for one or more populations that participated in clinical studies. To illustrate, the outcomes data 110 can indicate mortality of individuals that participated in clinical studies. In other illustrative examples, the outcomes data 110 can indicate occurrences of biological conditions, such as stroke or myocardial infarction.

At 112, the framework 100 can include deriving models from the clinical study data 102. The models can be included in model data 114 that can be evaluated according to implementations described herein. In various implementations, the models can be stored in one or more databases. The models can be accessed online and retrieved manually, in some cases, or via an automated process in other situations. The model data 114 can include information directed to the models derived from the results of the individual clinical studies. The models can represent a series of assumptions about the progression of a biological condition being studied in a clinical study for the population that participated in the clinical study. In some cases, the model data 114 can indicate a probability of a transition between states of a disease. In a particular example, the model data 114 can indicate a probability of an individual included in a certain population moving from a state of no stroke to a state of stroke or a probability of an individual included in a certain population moving from no heart disease to myocardial infarction. In particular implementations, the model data 114 can include one or more equations that can be used to predict the progression of a biological condition.

At 116, the framework 100 can include evaluating models for a number of populations using a cooperative framework with some competitive elements. The models being evaluated can be obtained from the model data 114. In addition, the populations utilized to evaluate the models can be generated from the population data 108. In some cases, aggregated information obtained from each of the populations included in the population data 108 can be used to generate virtual populations that are used to evaluate the models. The evaluation of the models can include generating a number of virtual populations and running simulations based on the models and the virtual populations. The simulations can produce predictions of the progression of a biological condition with respect to each of the individuals included in the virtual populations. The progression of the biological condition for each individual included in the virtual populations can be determined by running the simulations over a number of years and determining the probability that the individual will progress to various states of the disease as the age of the individual increases.

In various implementations, the models can be evaluated according to a cooperative framework. The cooperative framework can include determining how the different models can work together and evaluating the fitness of the individual models based on the contributions of the individual models to the overall prediction of the progression of a biological condition. In some cases, the cooperative framework can include evaluating a linear equation that includes variables that represent each model being evaluated and a coefficient for each model that indicates the contribution of the corresponding model in predicting the progression of the biological condition. The linear equation can be optimized to determine the coefficients for the models. In particular implementations, gradient descent techniques can be utilized to determine the local minimum of the linear equation.

In the illustrative example of FIG. 1, the evaluation of the models using a cooperative framework can produce an aggregate model 118 with coefficients indicating the contribution of each individual model. The aggregate model 118 is represented as aA+bB+cC+dD, where A, B, C, D are functions that represent the individual models and a, b, c, d are the coefficients indicating the influence of the individual models A, B, C, and D on the prediction of the progression of a biological condition. In an illustrative implementation, models, A, B, C, and D can predict the progression of diabetes and the aggregate equation aA+bB+cC+dD can also be used to predict the progression of diabetes. Additionally, the coefficients a, b, c, d can sum to 1 and the individual coefficients can have values ranging from 0 to 1. The coefficients with values closer to 1 have more influence over the prediction of progression of a biological condition than coefficients with values closer to 0.

Observed outcomes from actual clinical studies that are included in the clinical study data 102 can be used to determine the coefficients for each model. That is, by comparing the predictions of the progression of a biological condition generated by the models being evaluated with actual observed outcomes, a fitness of each model for predicting the progression of the disease can be determined. The closer that the predictions of a model are to the observed outcomes, the greater the contribution of the individual model in the aggregate model.

In some instances, competitive aspects can also be incorporated into the framework 100. For example, certain initial conditions can be provided that are used in a first iteration of the aggregate equation 118 before the optimization of the aggregate equation 118. For example, the initial conditions can indicate values for individual coefficients of the aggregate equation 118. In particular implementations, different initial conditions for the evaluation of the aggregate equation 118 can produce different values for the coefficients of the aggregate equation 118 after the optimization process. To illustrate, a first coefficient can have a first value (e.g., 0.2) for a first set of initial conditions and the first coefficient can have a second value (e.g., 0.3) for a second set of initial conditions. The results of the optimization of the respective sets of initial conditions can be evaluated with respect to the outcomes 110 and then compared to one another. In this way, the fitness of the aggregate model 118 with regard to different sets of initial conditions can be evaluated with respect to one another and a set of values for the individual coefficients of the aggregate equation 118 having a best fitness can be determined.

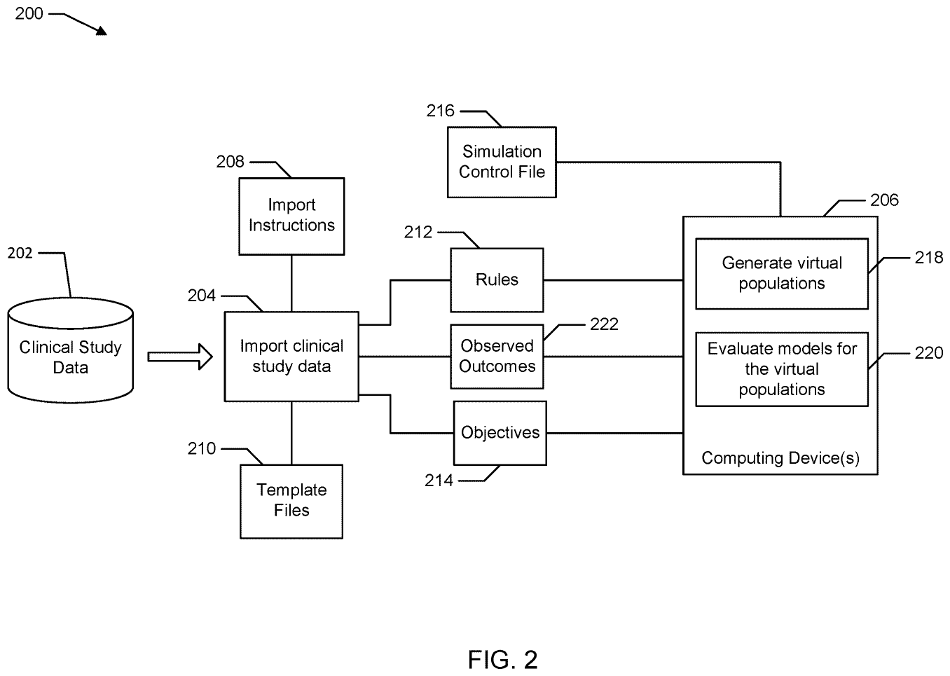

FIG. 2 includes a schematic diagram of a framework 200 for extracting information from clinical studies data to generate populations used to evaluate models that predict the progression of a biological condition. The framework 200 includes clinical study data 202 that is stored in one or more databases. In some cases, the clinical study data 202 can be similar to or the same as the clinical study data 102 of FIG. 1. In various implementations, the clinical study data 202 can be stored as extensible Markup Language (XML) data that can be parsed and extracted for use by various computing devices.

At 204, the framework 200 includes importing the clinical study data 202. In particular implementations, the clinical study data 202 can be imported to one or more computing devices 206. The one or more computing devices 206 can include software and/or one or more applications that can process the clinical study data 202 that has been imported. The clinical study data 202 can be imported utilizing import instructions 208 and/or template files 210. The import instructions 208 can include information used to obtain particular information from the clinical study data 202 such as population data, duration of clinical studies, inclusion/exclusion criteria, and data indicating the outcomes of the clinical studies. Other information can be extracted, as well, from the clinical study data 202 according to the import instructions 208, such as clerical information related to the clinical studies (e.g., description of the clinical study).

In some implementations, the import instructions 208 can be related to different phases of the process to import portions of the clinical study data 202. For example, in a first phase of data extraction, the import instructions 208 can filter the clinical studies obtained from the clinical studies data 202 in response to a query to obtain particular clinical studies data 202. In particular, the import instructions can extract titles of clinical studies, a description of the clinical studies, a duration of the clinical studies, and so forth, and provide this information to one or more template files 210. The template files 210 can store information obtained from the clinical studies data 202 in a particular format. In various situations, the template files 210 that include information obtained from the clinical studies data 202 in the first phase of data extraction can be analyzed to narrow the clinical studies from which to obtain data in subsequent phases of data extraction. To illustrate, a computing device or a computing device user can review a list of clinical studies produced during the first phase of data extraction to identify clinical studies to target in subsequent phases of data extraction based on a set of criteria.

In a second phase of importing clinical studies data 202, information from the subset of clinical studies identified in the first phase of information extraction is obtained. In the second phase of importing clinical studies data 202, the import instructions 208 are directed to extracting population information from the identified subset of clinical studies. The population information extracted from the clinical studies data 202 can include information that can be used to generate virtual populations that are used to evaluate the effectiveness of models associated with the clinical studies data 202. In some examples, the population information can include age, gender, physical characteristics (e.g., height, weight), dietary information, behavioral information (e.g., smoker/non-smoker, exercise habits), analyte levels (e.g., cholesterol level, HDL level, LDL level, triglycerides), other physical data (e.g., blood pressure, pulse rate), and so forth. The portions of the clinical study data 202 imported in the second phase of information importation can be stored in additional template files 210 that are designed to hold the population data.

Additionally, code can be generated for the population data extracted from the clinical studies data 202 indicated inheritance characteristics of population data. That is, inheritance code can indicate whether or not the information obtained with respect to a particular population can be used in conjunction with information obtained with respect to another population to generate a virtual population that can be used to evaluate models obtained from the clinical study data 202. For example, inheritance code generated in conjunction with the extraction of information from the clinical studies data 202 can indicate that weight and height information from one clinical study can be utilized in conjunction with age and triglyceride levels from another population to produce an aggregate virtual population.

Additional import instructions 208 can be utilized in a third phase of data importation to extract outcome data from the subset of clinical studies identified in the first phase of importing clinical studies data 202. In particular implementations, the import instructions 208 of the third phase of importing clinical studies data 202 are directed to extracting information from the clinical studies data 202 that indicates the states and/or characteristics of individuals that participated in the clinical studies. For example, the outcomes data for clinical studies related to heart disease may indicate the number of participants that suffered a heart attack in the duration of the clinical study and/or the number of participants that suffered a stroke during the clinical study. Previously observed outcomes extracted from the clinical studies data can be stored in particular template files 210 to be merged with newly extracted observed outcomes data 222 and used to validate the outcomes produced by models that are being evaluated.

In each phase of data extraction from the clinical studies data, the import instructions 208 and the template files 210 can differ. The template files 210 provide the extracted information in specific forms that are easily accessible and manipulatable by software executing on the computing devices 206 that is used to evaluate the models included in the clinical studies data 202.

In some implementations, the import instructions 208 can also include manipulation commands that process the extracted portions of the clinical studies data 202. The manipulation can include text processing commands. In particular implementations, the text processing commands can be related to handling Unicode and joining, replacing, and filtering text extracted from the clinical studies data 202. The import instructions 208 can also include conversion code that caused data extracted from the clinical studies data 202 to be converted into a standardized form. For example, the units for reporting levels of analytes in subjects can be different from clinical study to clinical study. In an illustrative example, the import instructions 208 can include code for converting mg/dL to mmol/L for HDL and triglycerides because the coefficients for this conversion can differ for HDL measurements and triglycerides measurements. In this way, the conversion of units can be flexible and context-aware. That is, based on the context of the values provided, certain conversion factors can be selected to produce the appropriate final values after the conversion takes place. The import instructions 208 can be used to modify, if necessary, information extracted from the clinical studies data 202 to match the standardized units of the import instructions 208 otherwise conversion will match the units in the template file 210. In another example, the import instructions 208 can include code for converting race and/or ethnicity information into a standardized format due to the variety of formats that clinical studies can report this type of information.

The import instructions 208 can also be utilized to generate code that can be utilized to generate individuals included in virtual populations that are used to evaluate models for predicting the progression of a biological condition. In some implementations, rules 212 and objectives 214 can be generated based on information obtained from the clinical studies data 202. The rules 212 and the objectives 214 can be used during the generation of virtual populations that can be utilized to evaluate models derived from the clinical study data 202. In some cases, the rules 212 can include parameters that can be utilized in generating virtual populations for models related to a particular biological condition. For example, the rules 212 can indicate that a virtual population is to include individuals within a certain age range and exclude individuals outside of that age range. In a particular illustrative example, the rules 212 can indicate that individuals under the age of 18 and over the age of 65 are not to be included in a virtual population. Additionally, the objectives 214 can indicate statistical distributions for a virtual population. To illustrate, the objectives 214 can indicate that a particular percentage of a virtual population is to have a level of an analyte within a specified range. In an illustrative situation, the objectives 214 can indicate that 50% of a virtual population is to have a blood pressure from 140 mmHg to 180 mmHg.

In some cases, the rules 212 and objectives 214 can be updated as new clinical studies are added to the clinical study data 202. In particular, as new clinical studies that satisfy the conditions of a query are added to the clinical studies data 202, the import instructions 208 can be implemented to import portions of the new clinical studies and store the newly imported information into the template files 210. The newly imported information can be stored in the template files 210 in conjunction with the information originally stored in the template files 210. In particular implementations, the rules 212 and the objectives 214 can also be modified to correspond with the changes to the clinical study data 202 brought about by the new information added to the clinical studies data 202.

A simulation control file 216 can also include information used to generate virtual populations and evaluate models indicating the progression of biological conditions. The simulation control file 216 can include information including the models to be evaluated, populations for the models to be evaluated against, and how to evaluate fitness of the models. The simulation control file 216 can also include inclusion/exclusion criteria for the model and population combinations to be simulated. Further, the simulation control file 216 includes instructions for coefficient optimization, such as stopping criteria (e.g., when to stop the optimization process), coefficient change methods and parameters between optimization iterations, and one or more initial conditions for optimization. The simulation control file 216 can also indicate that some coefficients can be static during the optimization process.

After obtaining the rules 212 and the objectives 214, the computing device(s) 206 can, at 218, generate one or more virtual populations. The virtual populations can include individuals that satisfy the rules 212 and the objectives 214. In particular implementations, the virtual populations generated by the computing device(s) 206 can have characteristics that correspond with the aggregate characteristics of actual populations studied in the clinical studies included in the clinical studies data 202.

At 220, the computing device(s) evaluate the models obtained from the clinical studies data 202 in light of the virtual populations generated at 218. That is, individual models obtained from the clinical studies data 202 are used to predict the progression of a biological condition for each individual included in the virtual populations. In particular implementations, simulations using the individual models are performed for the virtual populations to determine the outcomes for each individual with respect to the progression of a biological condition. The results of the simulations can be compared to the observed outcomes 222 that are obtained from the clinical studies data 202 to determine a fitness of a particular model to predict the progression of the biological condition.

In various implementations, each model is evaluated in light of multiple virtual populations. Additionally, multiple simulations can be run for each virtual population with respect to the individual models. In some cases, the fitness of a model to predict the progression of a biological condition can be determined using a cooperative framework where a number of models are evaluated together. The models can be evaluated by producing an aggregate model comprised of the individual models and determining the relative contributions of each individual model to the aggregate model.

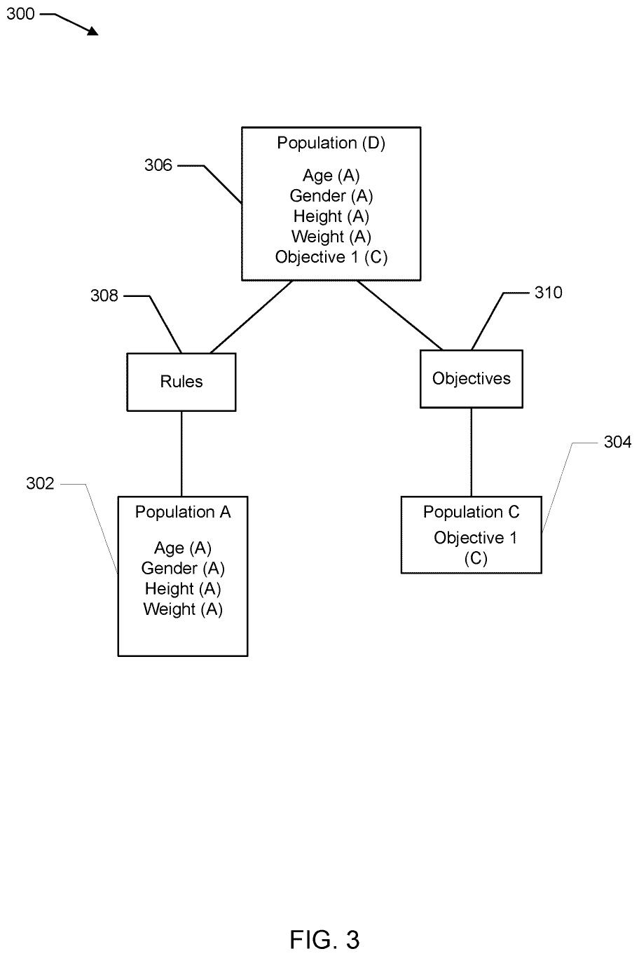

FIG. 3 includes a schematic diagram of a framework 300 showing the use of object oriented techniques to generate virtual populations used to verify models derived from clinical data. In particular, the framework 300 includes a first population object 302 corresponding to a first population and a second population object 304 corresponding to a second population. The first population and the second population can each relate to a group of individuals that participated in a clinical study. The population objects 302, 304 can include characteristics of the individuals included in the respective populations associated with the objects 302, 304. The characteristics can be represented by ranges, averages and standard deviations, distributions, combinations thereof, and the like. For example, the characteristics can be related to one another by arithmetic operations and other functions, such as one or more characteristics depending on gender or blood pressure. In the illustrative example of FIG. 3, the first population object 302 corresponds to the first population having characteristics corresponding to age, gender, height, and weight. Additionally, the second population object 304 corresponds to an objective of the second population. The objective relates to target values for a characteristic of a virtual population. To illustrate, an objective can indicate a mean and standard deviation for a characteristic, such as age, blood pressure, height, weight, etc. for a given virtual population.

The framework 300 also includes a third population object 306 that inherits rules 308 from the first population object 302 and objectives 310 from the second population object 304. The third population object 306 includes age characteristics, gender characteristics, height characteristics, and weight characteristic generated from the rules 308 associated with the first population object 302 and objective 1 inherited from the objectives 310 associated with the second population object 304.

In additional implementations, a population can inherit data from one or more additional populations. The data can include characteristics of individuals included in the one or more additional populations and can be extracted after generation of a population defined by rules and objectives. In some cases, the one or more additional populations can include individuals from at least one virtual population. In other situations, the one or more additional populations can include individuals from at least one actual population that participated in a clinical study. In various implementations, characteristics of an additional population can override one or more characteristics of another population, such as one or more characteristics of population A or population D. In these scenarios, the values of the characteristics (e.g., age, weight, height, etc.) of the additional population can replace the values of the characteristics of the original population. In particular implementations, characteristics of an additional population can fill in missing values of characteristics of a population. For example, population D does not include blood pressure information. In this situation, an additional population that includes blood pressure information can provide this information that is inherited by population D.

The ability for populations to inherit values of characteristics, objectives, or both from other populations provides flexibility in the generation of new populations that is not found in conventional population generation techniques. Further, the ability for populations to inherit values of characteristics, objectives, or both from other populations can lead to generating more complete populations by filling in missing data for some populations. In this way, populations can be generated that include characteristics that more closely correspond with the populations used to generate certain models. For example, if a model was generated from a population that measured HDL levels, but a population being used to evaluate the model does not include individuals with HDL data, the HDL levels of individuals from an additional population that includes values for HDL levels can be used to fill in the missing data. In this way, the framework of using object-oriented techniques to provide data to populations is different from conventional techniques that do not provide methods to fill in and substitute values for characteristics of populations.

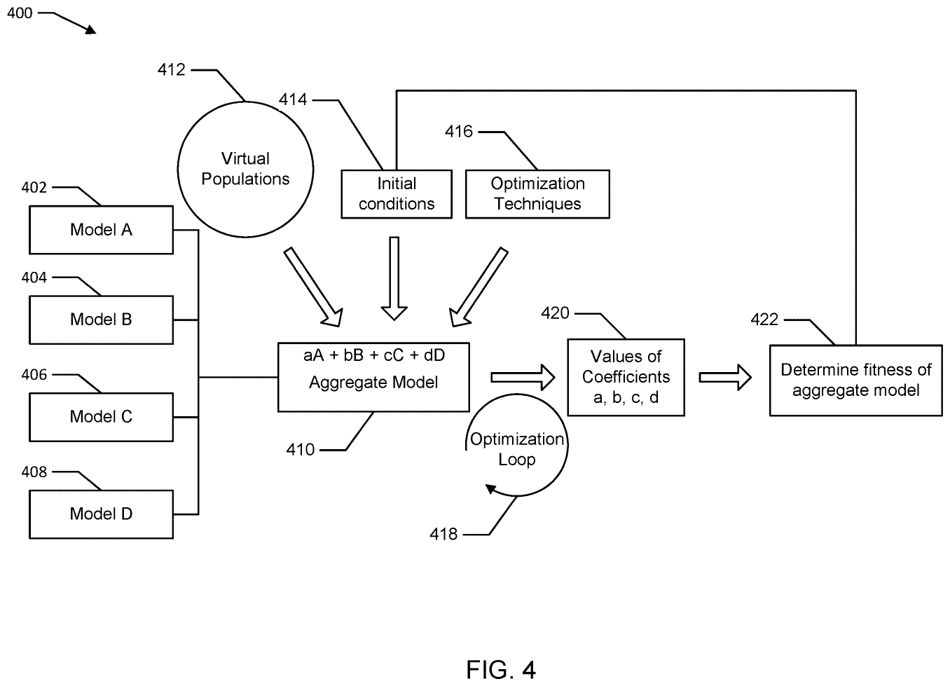

FIG. 4 shows a schematic diagram of a framework 400 to determine a combination of models that predicts progression of a biological condition. The framework 400 includes a first model 402, a second model 404, a third model 406, and a fourth model 408. The models 402, 404, 406, 408 can be derived from clinical data. In particular implementations, the models 402, 404, 406, 408 can be derived from clinical data corresponding to a particular biological condition such that the models 402, 404, 406, 408 can predict the progression of the biological condition. The framework 400 can determine the fitness of the combination of individual models 402, 404, 406, 408 in predicting the progression of the biological condition by evaluating an aggregate model 410. The aggregate model 410 can be a linear equation that includes variables corresponding to each model 402, 404, 406, 408 and coefficients a, b, c, and d, related to each model.

The aggregate model 410 can be evaluated using one or more virtual populations 412. The virtual populations 412 can be generated using information from populations that participated in the clinical studies used to produce the models 402, 404, 406, 408. In some cases, the virtual populations 412 can also be generated using information from populations other than those used to produce the models 402, 404, 406, 408, but corresponding to other clinical studies studying the progression of the same biological condition(s) as the clinical studies used to produce the models 402, 404, 406, 408.

In some implementations, the aggregate model 410 can be represented by the equation:

$$S ( {t}_{j} , {f}_{j} , {r}_{i} , {p}_{i} ) = {\sum }_{j} {g ( ( {t}_{j} ⊙ \{ {f}_{j} ( {p}_{i} ) + {e}_{ij} \} ) - g ( \{ r ( {p}_{i} ) \} ) )}^{2}$$

In this equation, s represents the fitness function that needs to be minimized, g represents the aggregate function and t is a term representing the model transformation. The models are represented by the term f and the virtual individuals that are being used to conduct the simulations are represented by p. A noise term is introduced with the variable e, while r represents the observed phenomenon from the clinical studies. The index i enumerates populations while the index j enumerates different models.

The aggregate model 410 can also be evaluated based on initial conditions 414. The initial conditions 414 can represent initial guesses regarding the coefficients for the different models included in the aggregate model 410. The initial conditions 414 regarding the coefficients can correspond to initial guesses of the starting points for contributions of the individual models in the evaluation of the aggregate model 410. The initial conditions 414 can also relate to the virtual populations 412. In these situations, the initial conditions 414 can indicate correlations between characteristics of individuals included in the virtual populations 412, such as increasing age corresponds to increasing blood pressure. When the initial conditions 414 relate to characteristics of the virtual populations 412, the initial conditions 414 can also indicate that values for a characteristic are static or not. Further, the initial conditions 414 can include inclusion/exclusion criteria for the virtual populations 412, a hamming distance, or both.

In addition, the aggregate model 410 can be evaluated using optimization techniques 416. The optimization techniques 416 can correspond to one or more algorithms that can be used to solve the linear equation associated with the aggregate model 410 to determine the fitness of the models 402, 404, 406, 408 in predicting the progression of the biological condition. In some cases, the optimization techniques can include gradient descent techniques. In other instances, the optimization techniques can include evolutionary computation techniques. In particular implementations, the optimization techniques 416 can be directed to finding a local minimum that solves the linear equation of the aggregate model 410. In some cases, the local minimum can be determined after performing multiple iterations using the optimization techniques 416 in an optimization loop 418. The number of iterations included in the optimization loop 418 can correspond to a stopping criteria.

In particular implementations, the stopping criteria can be a specified number of iterations, while in other situations, the stopping criteria can correspond to a value of a coefficient or other specified criteria. At the local minimum, the values of the coefficients 420 can be determined. The values of the coefficients 420 can indicate a contribution of the respective models 402, 404, 406, 408 to predicting the progression of the biological condition. For example, the aggregate model 410 can be solved and the values of the coefficients 420 can be a=0.32, b=0.39, c=0.20, and d=0.09. The values for the coefficients can indicate the models that are the most dominant or most influential in determining outcomes for a given combination of model. In the illustrative example, model B can be identified as the model that is the most influential in determining outcomes for the aggregate model 410.

The process of evaluating the aggregate model 410 can continue at 422 by determining the fitness of the aggregate model 410 with the values of the coefficients 420. The fitness of the aggregate model 410 can be determined by comparing the results of the simulations with observed outcomes for a similar population. In some implementations, at least a portion of the simulations can be performed concurrently. The differences between the results of the simulations for each equation and the observed outcomes can be used to determine a fitness score for the initial iteration. Simulations for aggregate model 410 can then be performed for the subsequent guess combinations for the transformation parameters and the corresponding fitness scores can be determined based on the differences between the simulation results and the observed outcomes. If the fitness scores improve, that is if the difference between the simulations and the observed outcomes decreases, then the iterative process can continue with guesses in a similar direction until one or more criteria are satisfied.

In particular implementations, the transformation parameters/coefficients can be static, variable, scaled, and/or normalized. In some cases, groups of transformation parameters can be of the same type. For example, a first group of transformation parameters can be static, while another group of transformation parameters can be variable. The transformation parameter groups can be formed, in some situations, based on a condition associated with a state of a biological condition. For example, a first group of transformation parameters/coefficients can be associated with disease states related to coronary heart disease for individuals with diabetes, while a second group of transformation parameters/coefficients can be associated with disease states related to stroke for individuals with diabetes. In various implementations, the transformation parameter groups can be associated with various inclusion criteria, exclusion criteria, and Hamming distance criteria. That is, a first group of transformation parameters can be defined by a first set of criteria, while a second group of transformation parameters can be defined by a second set of criteria. In some situations, the transformation parameters included in each group can change as the iterative process to solve the transformation proceeds.

During the iterative process to optimize the aggregate model 410, the values of the static type transformation parameters will remain constant. Additionally, if a transformation parameter falls outside of one or more of the criteria during one or more iterations of the optimization process, the value of the transformation parameter can be truncated to stay within each of the optimization criteria. In situations where a transformation parameter is a scaled transformation parameter, during the individual optimization steps, the scaled transformation parameters can be divided by the sum of the parameters and multiplied by a scaling factor. The scaling factor can be associated with the particular parameter group of the scaled transformation parameter. In other implementations, during the individual optimization steps, the scaled transformation parameters can be divided by the norm of the sum of the parameters and multiplied by a normalizing value. The normalizing value can be associated with the particular parameter group of the scaled transformation parameter.

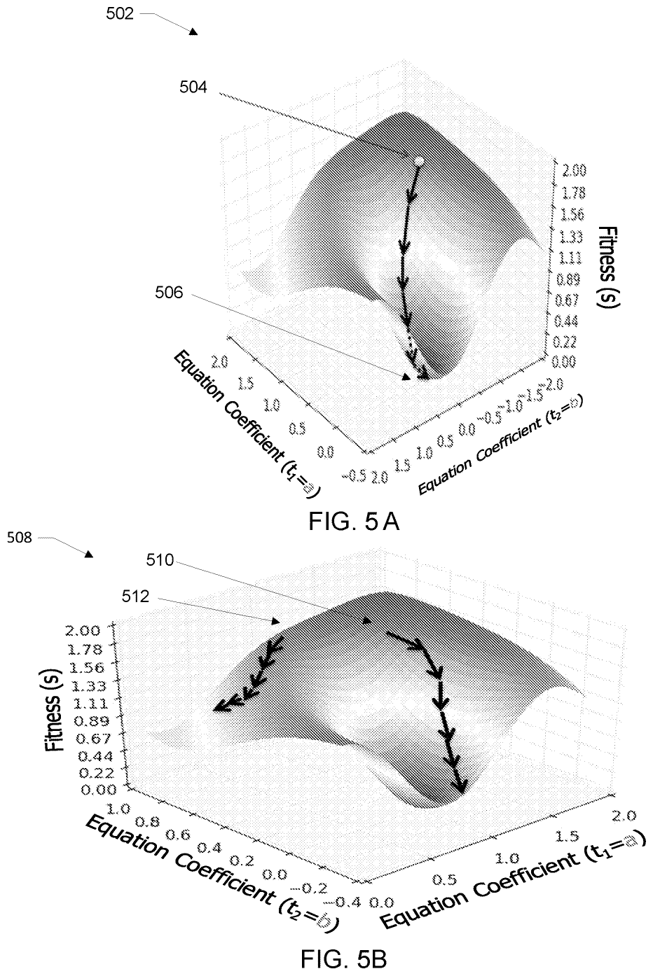

FIG. 5A shows an example implementation 502 of using gradient descent techniques to determine a local minimum for an aggregate fitness function that identifies the optimal contributions of each individual model to the aggregate fitness function, while FIG. 5B shows an example of using multiple initial guesses for the optimization process. The gradient descent technique provides cooperative features to determine an amount of contribution of each model included in an aggregate model. With each iteration of the gradient descent algorithm, the solution moves closer to a local minimum. The gradient descent algorithm can start at 504 and work towards 506. The use of gradient descent optimization techniques allows the optimal combination of multiple models to be determined in continuous parameter space rather than computing all model combinations in discrete parameter space, which reduces the processing resources and memory resources utilized to determine the aggregate model because the resources simply increase linearly per parameter for each gradient descent iteration as more equations are added rather than close to exponentially

The second example 508 included in FIG. 5B shows a number of initial guesses 510, 512 that can be evaluated. For each initial guess 510, 512, a gradient descent algorithm can be used to determine a local minimum. The use of the gradient descent algorithm to identify the local minimum can correspond to cooperative elements of the implementations described herein. The fitness of the end result of the coefficients determined for the local minima for each initial guess 510, 512 can be evaluated with respect to each other. The evaluation of the differing coefficients with respect to observed outcomes for each initial guess 510, 512 can represent certain competitive aspects of the implementations described herein.

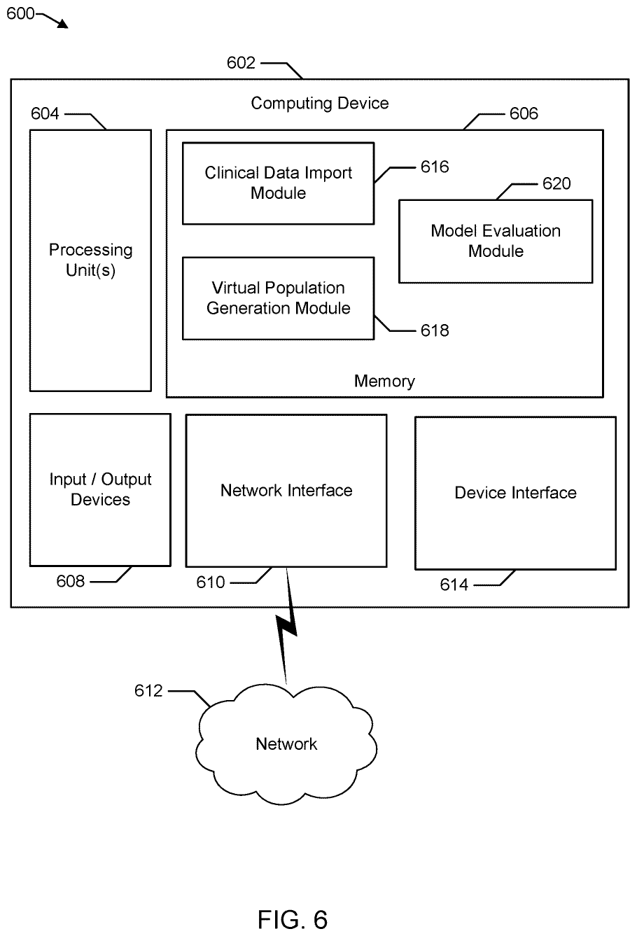

FIG. 6 shows a block diagram of an example computing device 600 to evaluate models derived from clinical data using a cooperative framework with some competitive elements. The computing device 602 can be implemented with one or more processing unit(s) 604 and memory 606, both of which can be distributed across one or more physical or logical locations. For example, in some implementations, the operations described as being performed by the computing device 602 can be performed by multiple computing devices. In some cases, the operations described as being performed by the computing device 602 can be performed in a cloud computing architecture.

The processing unit(s) 604 can include any combination of central processing units (CPUs), graphical processing units (GPUs), single core processors, multi-core processors, application-specific integrated circuits (ASICs), programmable circuits such as Field Programmable Gate Arrays (FPGA), and the like. In one implementation, one or more of the processing units(s) 604 can use Single Instruction Multiple Data (SIMD) parallel architecture. For example, the processing unit(s) 604 can include one or more GPUs that implement SIMD. One or more of the processing unit(s) 604 can be implemented as hardware devices. In some implementations, one or more of the processing unit(s) 604 can be implemented in software and/or firmware in addition to hardware implementations. Software or firmware implementations of the processing unit(s) 604 can include computer- or machine-executable instructions written in any suitable programming language to perform the various functions described. Software implementations of the processing unit(s) 604 may be stored in whole or part in the memory 606.

Alternatively, or additionally, the functionality of computing device 602 can be performed, at least in part, by one or more hardware logic components. For example, and without limitation, illustrative types of hardware logic components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc.

Memory 606 of the computing device 602 can include removable storage, non-removable storage, local storage, and/or remote storage to provide storage of computer-readable instructions, data structures, program modules, and other data. The memory 606 can be implemented as computer-readable media. Computer-readable media includes at least two types of media: computer-readable storage media and communications media. Computer-readable storage media includes volatile and non-volatile, removable and non-removable media implemented in any method or technology for storage of information such as computer-readable instructions, data structures, program modules, or other data. Computer-readable storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other non-transmission medium that can be used to store information for access by a computing device.

In contrast, communications media can embody computer-readable instructions, data structures, program modules, or other data in a modulated data signal, such as a carrier wave, or other transmission mechanism. As defined herein, computer-readable storage media and communications media are mutually exclusive.

The computing device 602 can include and/or be coupled with one or more input/output devices 608 such as a keyboard, a pointing device, a touchscreen, a microphone, a camera, a display, a speaker, a printer, and the like. Input/output devices 608 that are physically remote from the processing unit(s) 604 and the memory 606 can also be included within the scope of the input/output devices 608.

Also, the computing device 602 can include a network interface 610. The network interface 610 can be a point of interconnection between the computing device 602 and one or more networks 612. The network interface 610 can be implemented in hardware, for example, as a network interface card (NIC), a network adapter, a LAN adapter or physical network interface. The network interface 610 can be implemented in software. The network interface 610 can be implemented as an expansion card or as part of a motherboard. The network interface 610 can implement electronic circuitry to communicate using a specific physical layer and data link layer standard, such as Ethernet or Wi-Fi. The network interface 610 can support wired and/or wireless communication. The network interface 610 can provide a base for a full network protocol stack, allowing communication among groups of computers on the same local area network (LAN) and large-scale network communications through routable protocols, such as Internet Protocol (IP).

The one or more networks 612 can include any type of communications network, such as a local area network, a wide area network, a mesh network, an ad hoc network, a peer-to-peer network, the Internet, a cable network, a telephone network, a wired network, a wireless network, combinations thereof, and the like.

A device interface 614 can be part of the computing device 602 that provides hardware to establish communicative connections to other devices. The device interface 614 can also include software that supports the hardware. The device interface 614 can be implemented as a wired or wireless connection that does not cross a network. A wired connection may include one or more wires or cables physically connecting the computing device 602 to another device. The wired connection can be created by a headphone cable, a telephone cable, a SCSI cable, a USB cable, an Ethernet cable, Fire Wire, or the like. The wireless connection may be created by radio waves (e.g., any version of Bluetooth, ANT, Wi-Fi IEEE 802.11, etc.), infrared light, or the like.

The computing device 602 can include multiple modules that may be implemented as instructions stored in the memory 606 for execution by processing unit(s) 604 and/or implemented, in whole or in part, by one or more hardware logic components or firmware. The memory 606 can be used to store any number of functional components that are executable by the one or more processors processing units 604. In many implementations, these functional components can comprise instructions or programs that are executable by the one or more processing units 604 and that, when executed, implement operational logic for performing the operations attributed to the computing device 602. Functional components of the computing device 602 that can be executed on the one or more processing units 604 for evaluating models that predict the progression of a biological condition, as described herein, include a clinical data import module 616, a virtual population generation module 618, and a model evaluation module 620. One or more of the modules, 616, 618, 620 can be used to implement frameworks 100, 200, 300, 400, of FIG. 1, FIG. 2, FIG. 3, FIG. 4, and produce the examples of FIG. 5A and FIG. 5B.

The clinical data import module 616 can include computer-readable instructions that when executed by the one or more processing units 604 cause the computing device to extract data about one or more clinical studies from at least one database. In some cases, the database can be a private database maintained by one or more entities, such as an insurance company, a university, a health provider, combinations thereof, and so forth. In other situations, the database can be a public database maintained by one or more entities, such as a governmental entity. In an illustrative example, the database can include the website clinicaltrials.gov. The information stored in the one or more databases can include summary information for populations that have participated in clinical studies. The summary information can include values, such as mean, median, average, and the like, for different characteristics of a population (e.g., age, weight, cholesterol level, etc.). In particular implementations, the one or more databases may include more individualized information about the population, while still protecting the privacy of the individuals. For example, the databases can include information indicating a number of individuals of a particular age or a number of individuals of a particular weight.

The data obtained from the one or more databases can also include outcomes data that indicates the results of the clinical studies. The results of the clinical studies can indicate summary data and/or individualized data regarding the progression of biological conditions of individuals that participated in the clinical studies. The outcomes data can, in some cases, indicate a number of individuals that meet criteria for one or more biological conditions and/or that meet criteria for a state of a biological condition. For example, the outcomes data can indicate a number of individuals that suffered a stroke, a number of individuals that died during the clinical study, a number of individuals that have blood pressure within a specified range, and the like.

After obtaining information from the one or more databases, the clinical data import module 616 can filter the information according to one or more criteria. The one or more criteria can be included in a query of the extracted data. In particular implementations, the data can be filtered according to import instructions that modify the data extracted from the clinical studies database(s). In some situations, the data extracted from the database can be filtered and the data can be formatted according to particular templates. In additional implementations, conversion factors can be utilized that convert data from one set of units to another set of units. In various implementations, the instructions utilized to filter data extracted from a clinical studies database can be modified for filtering information from clinical studies that correspond to different biological conditions. Also, some features of previously utilized instructions can be re-used to optimize the resources utilized to filter the clinical studies information. In illustrative implementations, the instructions utilized to filter data obtained from a clinical studies database can modify the data such that the data can be utilized by algorithms, techniques, and engines that evaluate models that predict the progression of biological conditions.

The virtual population generation module 618 can include computer-readable instructions that when executed by the one or more processing units 604 cause the computing device 602 to generate one or more virtual populations. A virtual population can include characteristics of each individual included in the virtual population. For example, each individual of a virtual population can have a height, a weight, an age, a gender, a blood pressure, a cholesterol level, and so forth. The virtual population generation module 618 can utilize population summary data obtained from the clinical study data to generate specific information for each individual included in the virtual population.

In some cases, the virtual population generation module 618 can implement object oriented techniques in regard to the generation of a virtual population. For example, the virtual population generation module 618 can obtain instructions indicating that a virtual population is to be generated that derives characteristics from additional populations. To illustrate, a virtual population can be generated that derives a first set of characteristics from a first population and a second set of characteristics from a second population. In particular implementations, the first population and the second population can be other virtual populations, actual populations, or a combination thereof. In illustrative implementations, objectives, such as average blood pressure and a corresponding standard deviation or upper and lower blood pressure limits, can be provided by a population. To meet objectives provided by one or more populations, the virtual population generation module 618 can produce a number of virtual individuals that have certain characteristics and then filter the number of virtual individuals to produce a smaller population that meets the objectives as close as possible within computing constraints. Thus, if a rule or an objective indicates that the age range for the virtual population is to be from 45 to 79, the virtual population generation module 618 can remove any virtual individuals that have ages outside of the specified age range. In a particular illustrative implementation, the virtual population generation module 618 can choose a set of virtual individuals that best meet the objectives provided, such as the best 1000 virtual individuals out of 10,000 virtual individuals generated by the virtual population generation module 618.

The model evaluation module 620 can include computer-readable instructions that when executed by the one or more processing units 604 cause the computing device 602 to evaluate models that predict the progression of one or more biological conditions. The model evaluation module 620 can obtain one or more models that predict the progression of a biological condition. The one or more models can be produced from clinical study data. The model evaluation module 620 can utilize cooperative techniques to determine a fitness of a combination of the models. For example, an aggregate model predicting the progression of a biological condition can be produced from a plurality of models. In some cases, the aggregate model can be represented by an equation. In a particular illustrative example, the aggregate model can be represented by a linear equation having functions that correspond to each individual model of the aggregate model and a respective coefficient that corresponds to each function.

The model evaluation module 620 can evaluate the aggregate model with respect to at least one virtual population generated by the virtual population generation module 618. In various implementations, the model evaluation module 620 can utilize one or more algorithms to determine the values for the functions represented in the aggregate model. In a particular example, the model evaluation module 620 can utilize a gradient descent algorithm to identify a local minimum and identify the values of the functions for each model at the local minimum. The values of the functions can indicate a contribution or importance of each model of the aggregate equation. In some situations, a number of iterations of the gradient descent algorithm can be performed by the model evaluation module 620 to determine the local minimum for the aggregate model with each iteration getting closer to the local minimum.

The fitness of a particular combination of models included in the aggregate models and based on a set of coefficients can be used to determine outcomes for a virtual population. In illustrative implementations, the outcomes for the virtual population can be determined by evaluating the individuals included in the virtual population on a yearly basis and tracking the progression of a biological condition until the death of the virtual individuals caused either by a particular biological condition being studied or mortality caused by another biological condition. In particular implementations, the virtual population can correspond to an actual population that was used to derive at least one of the models included in the aggregate model. In some cases, the virtual population can correspond to a combination of actual populations that were used to produce the models of the aggregate model. The model evaluation module 620 can evaluate the fitness of the particular combination of models by comparing the simulated outcomes from the aggregate model and the virtual population with actual outcomes from a clinical study. In some implementations, multiple runs can be performed for an aggregate model and a corresponding virtual population to determine consistency between the outcomes for the aggregate model.

In various implementations, the models of the aggregate model can be evaluated using a set of initial conditions. The set of initial conditions can include initial guesses for the coefficients of each model. The set of initial conditions can also indicate constraints for the virtual population being generated. The set of initial conditions can also indicate assumptions or hypotheses to be evaluated, such as the effects that one characteristic of an individual (e.g., age) can have on another characteristic (e.g., cholesterol). The model evaluation module 620 can evaluate an aggregate model under a number of sets of initial conditions to determine the viability of various assumptions or hypotheses being tested using the aggregate model. For example, the initial conditions can include a hypothesis that treatment options for a biological condition improve outcomes over time. Continuing with this example, the aggregate model can be evaluated when the hypothesis is true and when the hypothesis is false. The outcomes of the evaluation of the aggregate model can be compared to actual outcomes to determine the viability of the hypothesis. To illustrate, the hypothesis that outcomes are improved as time progresses due to improved treatments over time can be more likely when the simulated outcomes are closer to the actual outcomes than the simulated outcomes when the assumption is not factored into the results.

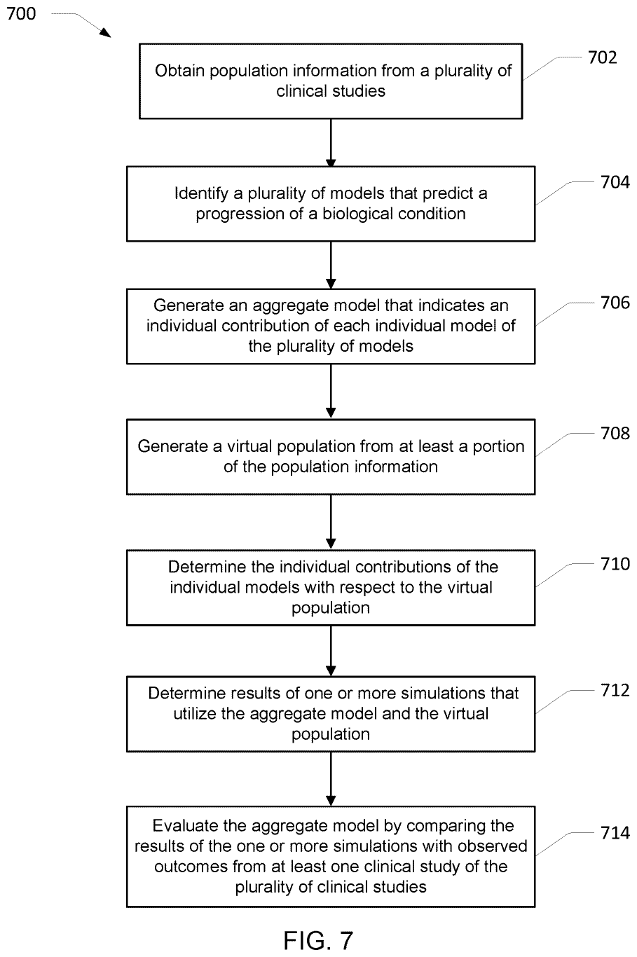

FIG. 7 is a flow diagram of an example process 700 to evaluate models derived from clinical data using a cooperative framework with some competitive elements. The operations illustrated in the example flow diagram of FIG. 7 can be implemented in hardware, software, or a combination thereof. In the context of software, the blocks can represent computer-executable instructions stored on one or more computer-readable media that, when executed by one or more processors, perform the operations recited in the blocks of the example flow diagram. The order in which the operations are described should not be construed as a limitation. Any number of the described blocks can be combined in any order and/or in parallel to implement the process 700, or alternative processes, and not all of the blocks need be executed.

At 702, the process 700 includes obtaining population information from a plurality of clinical studies. In some situations, the population information can be obtained from an online database. The population information can include summary information for one or more populations. The summary information can include at least one statistical measure for at least one characteristic of the one or more populations. For example, the summary information can include a mean, median, mode, average, a specific number, a proportion, a statistical distribution (e.g., 25 percentile) of a characteristic of a population, such as blood pressure, cholesterol level, height, etc.

In particular implementations, after extracting the population information from the online database, the population information can be filtered. In various implementations, the population information can be filtered according to a query to produce filtered population information. In additional implementations, the query can be included in import instructions that are used to filter the population information. In certain implementations, the filtered population information can be formatted according to a predetermined template to produce formatted population information. The formatted population information can be merged with prior population information stored in a template file. For example, the template file can include information that had been previously extracted from the online database corresponding to a different population that participated in a different clinical study.

In particular implementations, the formatting of the population information can be related to units of measurement of characteristics of individuals included in populations that participated in the clinical studies. For example, the population information can include values of a first characteristic related to the biological condition where the values are associated with a first unit of measurement. The values of the first characteristic can be converted from the first unit of measurement to a second unit of measurement. In some cases, the conversion from the first unit to the second unit can be specified by instructions used to obtain the population data. Additionally, the population information can include additional values of a second characteristic related to the disease where the additional values are associated with a third unit of measurement. The additional values of the second characteristic can be converted from the third unit of measurement to the second unit of measurement. In particular implementations, the first characteristic can have a first rate of conversion from the first unit of measurement to the second unit of measurement and the second characteristic can have a second rate of conversion from the third unit of measurement to the second unit of measurement. In an illustrative example, HDL levels can be converted from mg/dL to mmol/L using a first rate of conversion and triglycerides can be converted from mg/dL to mmol/L using a second rate of conversion.

At 704, the process 700 includes identifying a plurality of models that predict a progression of a biological condition. For example, the plurality of models can include a first model that is derived from at least one first clinical study and a second model that is derived from at least one second clinical study. The progression of the disease can include a plurality of states. In some cases, the progression of the disease can end in death.

At 706, the process 700 includes generating an aggregate model that indicates an individual contribution of each individual model of the plurality of models. The aggregate model can include an equation that corresponds to the individual models of the plurality of models and each model is associated with a value that indicates the contribution of the individual model.

At 708, the process 700 includes generating a virtual population from at least a portion of the population information. In some implementations, generating the virtual population can implement object-oriented techniques. For example, generating the virtual population can include generating a first object that includes first one or more rules related to determining values of characteristics of and includes first one or more objectives defining statistics for a first population of the plurality of populations. Additionally, generating the virtual population can also include generating a second object that includes one or more second rules related to determining values of characteristics and includes one or more second objectives defining statistics related to a second population of the plurality of populations. In these situations, the virtual population can include an object that inherits from the first object and the second object.

In various implementations, the object-oriented techniques can be utilized when conflicts arise between rules and/or objectives included in the particular objects utilized to generate the virtual population. The objectives can specify values for statistics of individuals included in the virtual population. To illustrate, a conflict can be determined between at least one first rule of the first object and at least one second rule of the second object. In other scenarios, a conflict can be determined between at least one first objective of the first object and at least one second objective of the second object. In a particular illustrative example, generating the virtual population can include generating a plurality of virtual individuals that satisfy one or more of: a particular first rule that does not conflict with at least one of the one or more second rules; a particular first objective that does not conflict with at least one of the one or more second objectives; at least one second rule that conflicts with at least one first rule; or at least one second objective that conflicts with at least one first objective. objectives that specify values for statistics of individuals included in the virtual population.

In an illustrative example, a virtual population object can be comprised of a first object that includes a first rule indicating that the age of virtual individuals is to be from 20 to 30 and a second object that includes a second rule indicating that the age of virtual individuals is to be from 25 to 35. The virtual population object can indicate that the second object supersedes the first object. In the case of this conflict, a virtual population is generated with virtual individuals having ages from 25 to 35.

Additionally, a virtual population object can be comprised of a first object that includes a first objective indicating that virtual population is to have a mean age of 25 and a second object that includes a second objective indicating that the virtual population is to have an average age of 32. The virtual population object can also indicate that the second object supersedes the first object. In the case of this conflict, a virtual population is generated with virtual individuals having an average age of 32.

A virtual population object can also inherit specific data for virtual individuals. For example, a virtual population object can be comprised of an object that includes particular ages of individuals, such as 22, 22, 23, 24, 24, 24, 25, 25, 26, 28, etc. In these situations, the virtual individuals of the virtual population have the same ages as the individuals included in the object from which the virtual population object inherits age data.

Object oriented techniques can also be used when virtual individuals of the virtual population are missing values for a characteristic. For example, an object can be identified that includes individuals having particular values of the characteristic. The virtual individuals of the virtual population can then be modified to have at least a portion of the particular values of the characteristic included in the object.

At 710, the process 700 includes determining the individual contributions of the individual models with respect to the virtual population. In some cases, the individual contributions of the individual models can be determined by optimizing the aggregate model using cooperative techniques. In certain implementations, determining the individual contributions of the individual models with respect to a plurality of virtual populations can include determining a local minimum of the aggregate model for the plurality of virtual populations. The local minimum, in various implementations, can be determined using a gradient descent algorithm such that the individual models cooperate during optimization and that is implemented over a number of iterations.

At 712, the process 700 includes determining results of one or more simulations that utilize the aggregate model and the virtual population. In some cases, the results of the one or more simulations are determined using a first set of initial conditions and additional results of one or more additional simulations can be determined that utilize the aggregate model, the virtual population, and that use a second set of initial conditions. The first set of initial conditions can include first estimates of the individual contributions of the individual models of the plurality of models, a first hypothesis, a first relationship between characteristics related to the biological condition, or a combination thereof. Additionally, the second set of initial conditions can include second estimates of the individual contributions of the individual models of the plurality of models, a second hypothesis that is a complement of the first hypothesis, a second relationship between characteristics related to the biological condition, or a combination thereof. In an illustrative implementation, the first hypothesis can be directed to an assumption that treatment for the biological condition improves over time, while the complement to the first hypothesis is directed to an assumption that treatment for the biological condition does not improve over time.

In some implementations, a first fitness of the first set of initial conditions can be determined based at least partly on first results of a first number of simulations for a plurality of virtual populations with regard to the observed outcomes. Also, a second fitness of the second set of initial conditions based at least partly on second results of a second number of simulations for the plurality of virtual populations with regard to the observed outcomes. The first fitness and the second fitness can be compared to evaluate the first set of initial conditions with respect to the second set of initial conditions.

At 714, the process 700 includes evaluating the aggregate model by comparing the results of the one or more simulations with observed outcomes from at least one clinical study of the plurality of clinical studies. The difference between the simulated outcomes and the observed outcomes can indicate the fitness of the aggregate model. In particular implementations, the greater the difference between the simulated outcomes and the observed outcomes, the less fit the aggregate model and the smaller the difference between the simulated outcomes and the observed outcomes, the more fit the aggregate model.

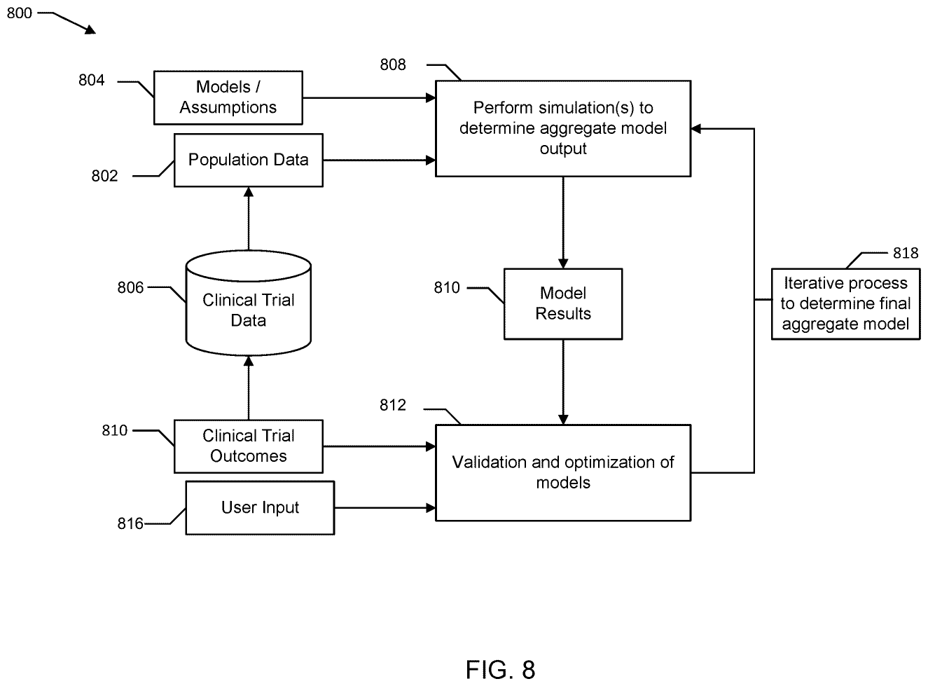

FIG. 8 is a block diagram of a framework 800 to incorporate user input into the process of generating aggregate models to predict the progression of a biological condition. The framework 800 can include clinical study data 802. The clinical study data 802 can be stored in one or more databases. The clinical study data 802 can be accessible by computing devices via an interface. In some cases, the interface can include a webpage that enables access to the clinical study data 802 being stored by the one or more databases. In other implementations, the clinical study data 802 can be accessed via a computing device application. In particular, the clinical study data 802 can be accessed using an app executing on a mobile computing device, such as a tablet computing device or a smartphone.

The clinical study data 802 can include information related to clinical studies that have been conducted by scientists and/or scientific organizations. The clinical studies can be related to various biological conditions. In some scenarios, the biological conditions can include diseases. In particular implementations, the biological conditions can be related to a level of an analyte present in subjects of the clinical studies. In some situations, the clinical studies can examine the effects of one or more factors on a biological condition. The factors can include characteristics of subjects participating in the clinical studies, such as age, weight, gender. The factors that can affect a biological condition can also include levels of analytes measured in subjects. For example, factors that can affect a biological condition can include cholesterol levels, triglyceride levels, HDL levels, LDL levels, and the like. Additionally, the factors that can affect a biological condition can include behaviors of subjects participating in clinical studies. To illustrate, the factors can include information related to diet (e.g., servings of fruits and/or vegetables per day), exercise, sleep, and so forth.

The framework 800 can also include a number of models 804. The models 804 can represent a series of assumptions about the progression of a biological condition being studied in a clinical study for the population that participated in the clinical study. In some cases, the models 804 can indicate a probability of a transition between states of a disease. In various examples, the models 804 can include equations extracted from the clinical study data 802 that indicate the probability of transition by individuals between states of a biological condition. In one or more illustrative examples, the models 804 can indicate a probability of an individual included in a certain population moving from a state of no stroke to a state of stroke or a probability of an individual included in a certain population moving from no heart disease to myocardial infarction. In particular implementations, the models 804 can include one or more equations that can be used to predict the progression of a biological condition. In one or more examples, the models 804 can be included in the clinical study data 802. In one or more additional examples, the models 804 can be obtained from sources outside of the clinical study data 802. In various implementations, the models 804 can be stored in one or more databases. The models 804 can be accessed online and retrieved manually, in some cases, or via an automated process in other situations. Additionally, the models 804 can be derived from the results of one or more clinical studies included in the clinical study data 802.

The framework 800 can also include population data 806. The population data 806 can include information related to the populations that participated in the individual clinical studies including baseline population distributions. In various examples, the population data 806 can include summary information for one or more populations. The summary information can include at least one statistical measure for at least one characteristic of the one or more populations. For example, the summary information can include a mean, median, mode, average, a specific number, a proportion, a statistical distribution (e.g., 25 percentile) of a characteristic of a population, such as blood pressure, cholesterol level, height, etc.

In various examples, the framework 800 can, at 808, include performing one or more simulations to determine an aggregate model and output of the aggregate model. The output of the aggregate model can include model results 810. In some cases, the model results 810 of the one or more simulations are determined using a first set of initial conditions and additional results of one or more additional simulations can be determined that utilize the aggregate model, one or more virtual populations, and that use a second set of initial conditions. The first set of initial conditions can include first estimates of the individual contributions of the individual models of the plurality of models, a first hypothesis, a first relationship between characteristics related to the biological condition, or a combination thereof. Additionally, the second set of initial conditions can include second estimates of the individual contributions of the individual models of the plurality of models, a second hypothesis that is a complement of the first hypothesis, a second relationship between characteristics related to the biological condition, or a combination thereof. In an illustrative implementation, the first hypothesis can be directed to an assumption that treatment for the biological condition improves over time, while the complement to the first hypothesis is directed to an assumption that treatment for the biological condition does not improve over time.

The one or more virtual populations used to perform the one or more simulations can include a number of virtual individuals that are generated using the population data 806. In one or more examples, the virtual individuals included in the one or more virtual populations can be generated using summary data included in the population data 806. In these scenarios, the virtual individuals included in the one or more virtual populations may not correspond to actual individuals that participated in clinical studies. In one or more implementations, the one or more virtual populations used to perform the one or more simulations can be generated by the virtual population generation module 618 of FIG. 6.

The one or more simulations can determine one or more transitions between states of one or more biological conditions by virtual individuals. In one or more examples, the transitions made by virtual individuals can be determined based on the models 804 with respect to a period of time. The model results 810 can indicate a respective disease state of virtual individuals over a period of time. In various examples, the model results 810 can indicate a cause of death of virtual individuals in relation to one or more disease states related to the models 804 and/or with respect to other biological conditions that are not related to the models 804.

The one or more simulations can be performed with respect to a number of models 804 obtained from the same clinical study or from different clinical studies. For example, the simulations can be performed using a first equation from a first clinical study to represent the transition from a first disease state to a second disease state and using a second equation from a second clinical study to represent the transition from the second disease state to a third disease state. The one or more simulations can also be performed by determining a contribution of each of the models 804 to the model results 810 and performing the one or more simulations using the respective contributions of the individual models 804. In one or more illustrative examples, the one or more simulations can be performed using one or more Monte Carlo simulation techniques.

At 812, the framework can perform one or more validation processes and one or more optimization processes with respect to the models 804 in relation to one or more virtual populations and with respect to the model results. The validation of the models 804 can include determining a fitness of a set of initial conditions utilized with respect to the one or more simulations performed with respect to operation 808. In one or more illustrative examples, the initial conditions can include a group of models used to perform the one or more simulations, such as the respective models used to determine the transition states between disease conditions, and the contributions of the individual models utilized with respect to the one or more simulations. In one or more examples, the model results 810 can be analyzed with respect to clinical study outcomes 814 to determine a fitness for a set of initial conditions. In some implementations, a first fitness of the first set of initial conditions can be determined based at least partly on first model results of a first number of simulations for a plurality of virtual populations with regard to the clinical study outcomes 814. Also, a second fitness of the second set of initial conditions based at least partly on second model results of a second number of simulations for the plurality of virtual populations with regard to the clinical study outcomes 814. The first fitness and the second fitness can be compared to evaluate the first set of initial conditions with respect to the second set of initial conditions. In one or more implementations, the validation and optimization of models performed with respect to 812 can be performed by the model evaluation module 620 of FIG. 6.

The validation and optimization of models performed at 812 can also utilize user input 816. The user input 816 can include the input of individuals that can be considered experts with respect to one or more biological conditions related to the clinical study data 802 and the models 804. The validation and optimization of the models can include determining a fitness of the input from the individual experts. Weightings of the input from the individual experts can also be determined and evaluated. The fitness scores of the models, the fitness scores of the experts, the weightings of the models, and the weightings of the experts can then be evaluated together. In one or more examples, the validation and optimization of the models 804 and the user input 816 can be performed using one or more gradient descent algorithms. In one or more illustrative examples, the user input 816 can indicate a correlation between an outcome utilized during the one or more simulations and a reference outcome.

At 818, an iterative process can be performed to determine a final aggregate model. The final aggregate model can be generated after determining that convergence of a gradient descent algorithm.

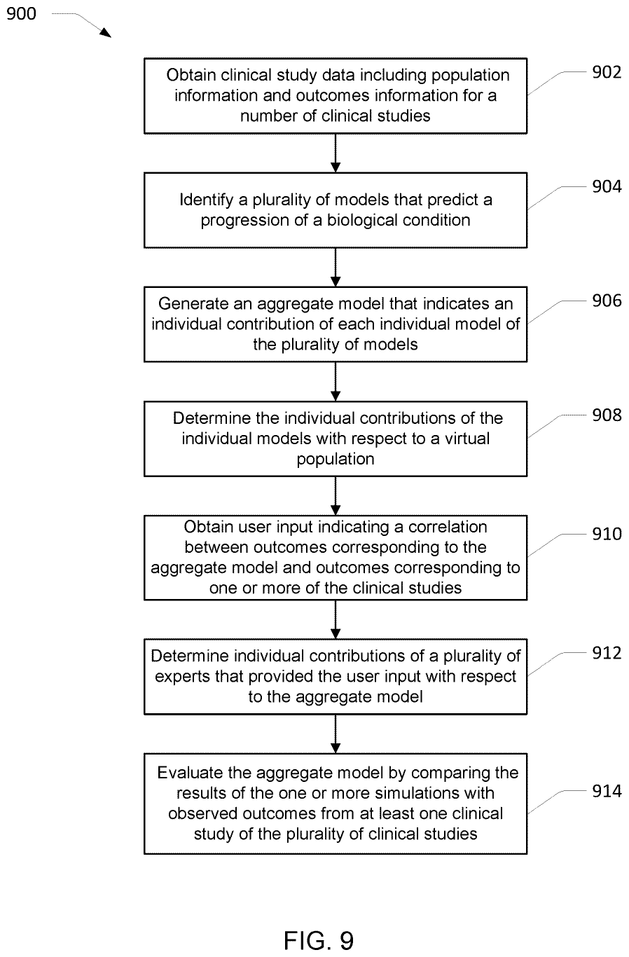

FIG. 9 is a flow diagram of an example process 900 to incorporate user input into generating an aggregate model to predict the progression of a biological condition. The process 900 can include, at 902, obtaining clinical study data including population information and outcomes information for a number of clinical studies. In some situations, the population information can be obtained from an online database. The population information can include summary information for one or more populations.

At 904, the process 900 can include identifying a plurality of models that predict a progression of a biological condition. For example, the plurality of models can include a first model that is derived from at least one first clinical study and a second model that is derived from at least one second clinical study. The progression of the disease can include a plurality of states. In some cases, the progression of the disease can end in death.

The process 900 can include, at 906, generating an aggregate model that indicates an individual contribution of each individual model of the plurality of models. The aggregate model can include an equation that corresponds to the individual models of the plurality of models and each model is associated with a value that indicates the contribution of the individual model.

In addition, at 908, the process 900 can include determining individual contributions of individual models with respect to a virtual population. In some cases, the individual contributions of the individual models can be determined by optimizing the aggregate model using cooperative techniques. In certain implementations, determining the individual contributions of the individual models with respect to a plurality of virtual populations can include determining a local minimum of the aggregate model for the plurality of virtual populations. The local minimum, in various implementations, can be determined using a gradient descent algorithm such that the individual models cooperate during optimization and that is implemented over a number of iterations.

Further, the process 900 can include, at 910, obtaining user input indicating a correlation between outcomes corresponding to the aggregate model and outcomes corresponding to one or more clinical studies. The user input can be obtained from a number of experts that evaluate definitions of outcomes related to clinical studies and the definitions of outcomes utilized when evaluating the aggregate model.

At 912, the process 900 can include determining individual contributions of a plurality of experts that provided the user input with respect to the aggregate model. The contributions of the individual experts can be determined when evaluated in conjunction with the evaluation of the aggregate model. For example, a fitness of the input provided by individual experts can be evaluated and used to determine the contribution of the input provided by the respective experts.

The process 900 can also include, at 914, evaluating the aggregate model by comparing the results of the one or more simulations with observed outcomes from at least one clinical study of the plurality of clinical studies. The aggregate model can be evaluated by determining fitness scores with respect to initial conditions evaluated in relation to the aggregate model. Additionally, the aggregate model can be evaluated in relation to the contribution of the respective experts.

### EXAMPLES

### Example 1

### Abstract

The COVID-19 pandemic has accelerated research worldwide and resulted in a large number of computational models and initiatives. Models were mostly aimed at forecast and resulted in different predictions as those were based on different assumptions. In fact the idea that a computational model is just an assumption attempting to explain a phenomenon has not been sufficiently explored. Moreover, the ability to combine models has not been fully realized.

The Reference Model for disease progression was been performing this task for years for diabetes models and recently started modeling COVID-19. The Reference Model is an ensemble of models that is optimized to fit observed disease phenomenon. The ensemble has the ability to include model component from different sources that compete and cooperate. The recent advance in this model is the ability to include models calculated in different scales making the model the first known multi scale ensemble model. This manuscript will review these capabilities and show how multiple models can improve our ability to comprehend the COVID-19 pandemic.

### Introduction

The impact of the COVID-19 pandemic was negative when considering the loss of life. However, it has some positive impact on technological development, it has stirred multiple groups to develop technologies to address the pandemic. Examples of positive organization are data collection group such as the Covid Tracking Project that collected data and made it available in a useful format, The Models of Infectious Disease Agent Study (MIDAS) and the Multiscale Modeling and Viral Pandemics working group associate with the Interagency Modeling and Analysis Group who coordinated scientists and made their work known and better accessible.

In the first half a year of the pandemic, many groups developed models that were already reported by the author. Those included variations on the SIR model based on differential equations, agent based models, and other models. The large amount of models was evident. and the CDC took action and assembled an ensemble model—the Covid-19 Forecast Hub that combined many models together to forecast mortality and hospitalization. This was the first attempt at accumulating knowledge systematically. However, it was limited to simple statistical aggregation-such as arithmetic average or median. This type of ensemble is simplified and leaves the validation task to the models and cannot identify the value of each model.

When the pandemic progressed and a vaccine was in sight, another group recommended an ensemble model approach that was much more sophisticated. This suggested approach was based on a technique previously used in where models were mixed with densities aimed at influenza. The sophisticated technique draws from base mathematical ideas published aimed at ensembles of Neural Networks. The new approach treats models as hypothesis that can be assembled together and can contribute an influence to the final result based on a density function and decides on level of influence. That function is decided using on machine learning techniques or optimization against known data. However, despite the idea this approach was not implemented fully on COVID-19 and only recommended. This approach, despite being innovative and applied an advanced mathematical technique to disease models, failed to acknowledge an already existing application of an ensemble disease model that used such advanced techniques at the time.

The Reference Model for disease progression was already an ensemble model modeling Diabetes at the time of publication of the techniques. The Reference Model existed since 2012 as model accumulating other models and creating a competition among themselves using High Performance Computing (HPC) with Microsimulations. The unique approach in this work allowed multiple competing and cooperating models to be bundled together and the ensemble was optimized using existing observed data on the disease. In the case of diabetes, model outcomes were compared to clinical studies, This Technology is now protected by 2 US patents.

With the start of the COVID-19 pandemic the modeling technology was adapted to handle infectious diseases. The Reference Model for COVID-19 was created with a simplified approach that did not show its full potential. This approach was recently enhanced to show more of its capabilities and construct the first multi scale ensemble model for COVID-19.

### Multi Scale Ensemble for COVID-19.

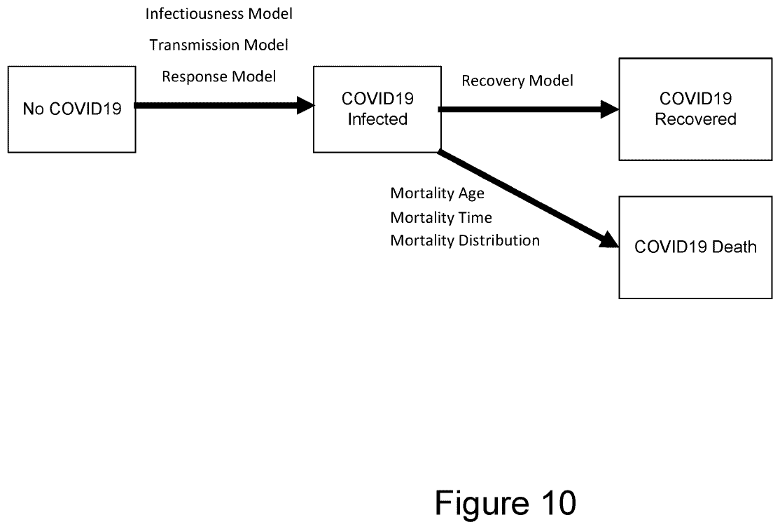

The basic structure of the model includes 4 states: No COVID19, COVID19 Infected, COVID19 Recovered, and COVID19 Death-see FIG. 10. This structure may resemble a simple SIR model while adding death, yet the model is much more sophisticated and includes many models and parameters. In fact, each transition in the diagram is controlled by multiple models-hence the ensemble model.

The transition probability between No COVID19 and COVID19 Infected states is controlled by 3 groups of models: Infectiousness Models: Indicating the level of infectiousness of each individual from time of infection. Note that the infectiousness of others effects the individual that is not infected and therefore not infectious. Transmission Models: indicating the probability of contracting the disease considering encounters with infected individuals. Response models: The behavior choice each individuals that affects the number of interactions in response to the pandemic and their own infectiousness state.

The transition into COVID19 Death state takes into account only deaths related to COVID-19. The simplifying assumption is that there is no competing mortality process out of other diseases in this model. Although COVID-19 mortality is roughly 10% of all mortality in the US, this assumption should not have a large impact on simulation since death is still a rare event and we assume our simulation censors individuals that died from other causes. The modeling technology used allows having multiple competing processes similar to how diabetes was modeled. However, the model was kept simple on purpose at this stage of development. Even death registered as COVID-19 death may have other factors such as another illness and modeling this requires modeling human interpretation. The mortality transition probability is composed of several models: Mortality Models: Mortality tables indicating the probability of dying from COVID-19 by age. Mortality Time: Models attempting to estimate the time of mortality since infection. Mortality distribution: A model that indicates the daily probability of mortality by age group since infection.

The transition into COVID19 Recovered state is one directional, indicating that is model does not include reinfection. Since the model was executed most of the population was still uninfected, this assumption is reasonable. Moreover, unlike the preliminary version of the model, the recovery numbers are not used in validation in this model. The Recovery model is a:
Recovery model: defines condition of recovery as a combination of infectiousness, mortality probability, mortality time and time since infection.

The Reference Model then executes all the above models and their variations and combines them to fit observed data. In this work we revisit the same observations provided by the COVID tracking project for 51 US states and territories over the period of two months since April 1st 2020, as reported on 9 Jun. 2020. The model results of numbers of infections and numbers of deaths are compared to the observed data and participate in the fitness score that is being optimized. Note that recoveries are no longer used as a reference in this work since some states did not report these. Moreover, in this work deaths are considered 1000 times more important than infections since deaths are more rare and we wish those to have effect, 2) death numbers are considered more reliable than infections due to questions regarding testing level and testing accuracy as well as testing strategy per state. Therefore infections are a factor we include in the fitness score showing the difference between model results and observed data.

The fitness score is then optimized using a variation of gradient descent to calculate the mixture of models and their influence on the ensemble. This process is repeated multiple times until convergence occurred and the mixture of models can be inspected.

One major importance in this work is the fact that the models that create the ensemble represent different phenomena and were computed using different scales. Infectiousness models were extracted from cell level and viral load models, individuals models derived from contact tracing, and population models, while the mortality models were extracted from population models and cell level models. Those models are how they are combined are explained hereafter:

### Model Combination:

The basic idea in an ensemble model is that each model potentially contributes to the results-indicated by its influence w, In this model, all models are organized in groups that model the same phenomenon, for example all infectiousness models are modeling the same attributes in the same terminology and for each group of models there are two rules: The influence of the model is positive-meaning that the models are not intentionally deceptive. This is modeled by w, for each model. The sum of contributions of each model in a model group sums to 1. This creates a competition between models since an increase of influence of one model means another model needs to give away influence. Please note that those rules apply for each group of models, and there are multiple groups. So the above constraints apply per group.

The influence of each model, can be realized in several ways: By influencing a quantity directly—for example a transition probability between states can be a sum of model contributions. By being applied to a proportion w of the individuals in a simulation randomly. Since simulation happens at the individual level, changing part of the population has an effect on the entire population result. Note that simulation results are aggregated. Combination/Nesting of the above two techniques, where a quantity that is combined by a group of models use computation of another model group that affect individuals and vice versa. Such combinations can be nested so that the contribution of model influences create complex functions that govern the simulation that are hard to define mathematically.

Note that this technique allows constructing ensemble models that are intelligible and can be comprehended by humans with modern machine learning models that are sometimes perceived more accurate, yet are harder to comprehend for a human and many times referred to as black boxes. The use of intelligible models has value of being able to explain things to a human is clearly understood if a researcher can follow the logic of a model. Constructing intelligible models and combining them among themselves and potentially with less intelligible modern machine learning models will not only allow better assessment of model value, it also allows measuring our comprehension of observed phenomenon. This also may have value in forums where court of law where models may be tried in the future where humans make decisions and need to assess model credibility towards a verdict.

Also note that constructions of models of different types together and formalizing the way that assumptions represented by models are plugged into the system opens new opportunities for modelers to construct models from components that can be assembled. together. In the future modelers can concentrate on the task focusing on modeling a smaller phenomenon while leaving modeling of larger tasks for modelers specializing in assembly of models using ensembles.

Once the base for model combination is explained it is possible can dive into specifics of implementation of the COVID-19 model.

### Initialization

This paper skips a lengthy discussion on how populations are generated for states as this aspect did not dramatically change from what was described before. In short a population for each state is generated to have all necessary parameters used in simulation for each state to match statistics as reported by The Covid Tracking project at the first day of simulation. Additional statistics are derived from US Census. Evolutionary computation is used to optimize the randomly generated individuals to match the target statistics.

After populations are generated the model computations can start. We will describe essence of computations while focusing on the models defined.

### Infectiousness:

During the pandemic, the DHS released a master question list about the pandemic This document updated regularly and evolved during the pandemic. The version from 26 May 2020 has the following question: “What is the average infectious period during which individuals can transmit the disease?” Clearly this was a question that was not answered for a while an although the document was pointing to some publications that may produce an answer, there was no conclusive answer. In fact at early stages of the pandemic, there were different speculations on the disease length.

The Reference Model first publications attempted to predict the disease duration through optimization in the absence of information for an early version of the model. However, the duration just captured the length until recovery while there are several periods in the disease: latency, infectiousness, and time till recovery. During development assumptions on infectiousness period were extracted from publications that include ranges of incubation periods, while taking the assumptions that the incubation period ranges represent infectiousness. The Reference Model allowed entering such assumptions with the absence of information and indeed some preliminary simulations included those models. Recall that the ensemble treats models as assumptions and balances those, so it is one possible use case-when there is little information. Initially there was one publication that described the infectiousness period and latency period. This was modeled as a period where the person is fully infectious from start period to end period and considered as Model 1. With time passing, more publications appeared that calculate the infectiousness period:

A model calculating viral load at the upper and lower respiratory track provided multi scale information from the cell and organ level while considering individual level information. The model provided several curves of infectiousness in that publication, two sub figures were digitized by hand and indicated the infectiousness level for each day. Model 2 was manually digitized from while numbers after day 15 were extrapolated manually by eye and represents a long lasting infectiousness period. Model 3 was manually digitized and represents a short infectiousness period. Model 4 was manually digitized from another figure. The curve was extracted and scaled so that max infectiousness is unity. At the end of the process, there were 4 infectiousness models that indicated relative infectiousness level per day since infection. The overall infectiousness level is a weighted combination of those functions using the influence of each model. This combination is quantitative. Note that infectiousness is only part of the construct and after it is computed, Infected interaction for the individual are calculated.

This quantity takes into account the fact that a person interacts with other individuals. For a person fully infectious and in the infected state, this number will match the number of interactions, yet for a person that is less than fully infectious this quantity is scaled down diminishing this quantity that indicated the contribution of this person to potential infections. This quantity is accumulated for all individuals in the simulation and forms an aggregate quantity called: InfectedInteractions. This quantity will be discussed when calculating response models and transmission models,

### Transmission Models

The transmission model considers 3 elements:
Individual Encounter—What is the probability of transmission in case infected individuals are encountered. The main coefficient there defines the probability of contracting the disease per one encounter with an infected person.
Population Density—How does this probability change with population density. This is controlled by a coefficient that indicates the relative population density boost to the encounters probability.
Random Constant—What is the probability of contracting the disease due to another reason other than direct contact with a modeled infectious person. For example, contracting the virus from a person outside the modeled group, such as a person visiting out of state falls into this group.

In this paper a basic form of equation is used for the transmission probability.

fThe logic behind this equation is explained in a paper published in Cureus titled as “The Reference Model Initial Use Case for COVID-19” where the coefficients were estimated as t 0.06 and b Coef PopDensity 0.1 In this work we reuse the same format while adding a random constant coefficient. We present 4 variations of those parameters to construct 4 different assumptions on transmission as presented in Table 1.

| Transmission | | | | |
|---|---|---|---|---|
| function # | Individual | Population | Random | |
| i | Encounter | Density | Constant | Comments/Rational |
| 1 | 0.5 | 0 | 1e-6 | Low bound - Similar |
| | | | | to previous publication |
| | | | | with slightly |
| | | | | lower a to represent |
| | | | | a low bound while |
| | | | | ignoring density |
| | | | | and adding a small c. |
| 2 | 10 | 0 | 4e-6 | Very high a that is |
| | | | | probably |
| | | | | unreasonable and |
| | | | | adding a higher |
| | | | | randomness. |
| | | | | This was added |
| | | | | on purpose to show |
| | | | | how unreasonable |
| | | | | assumptions are |
| | | | | treated in the |
| | | | | ensemble. |
| 3 | 1.5 | 0.1 | 0 | Reasonable |
| | | | | assumption - elevated |
| | | | | transmission |
| | | | | with original |
| | | | | population density. |
| 4 | 2.5 | 0.2 | 0 | Reasonable |
| | | | | assumption - more |
| | | | | elevated |
| | | | | transmission with |
| | | | | elevated population |
| | | | | density. |

In this paper a basic form of equation is used for the transmission probability:

Those assumptions are were selected after some trail an error. The first two models represent extremes bounds and the other two models represent reasonable assumptions considering that infectiousness period has been introduced reducing the number of days transmission occurs and hence the transmission per encounter should rise from the previous publication. Also a wider range of density population influence can be explored during optimization as was not easily done in the first publication.

Note that transmission probability depends on the proportion of population that is infected and their level of infection. This is possible by using the quantity InfectedInteraction previously calculated and deciding it by the total number of interactions.

Those assumptions are compose the transmission probability. The ensemble model contraction here is of a quantity. However, it is actually nested since it includes elements influenced by proportion of population as discussed in response models.

### Response Models

Unlike infection models and transmission models, response models do not contribute directly to a quantity in the ensemble. Instead each response models affects a proportion of the population associated with its weight in the ensemble. Response models are actually behavior models that decide on the number of interactions each person will have. The base number of interactions was extracted as described in the Cureus publication in a as a function of age. However, this number is modified in this aper as a function of the response scheme of each individual while adding assumptions on possible behavior of an individual according to their infectiousness state. Additional factors possibly influencing interactions in some response schemes are mobility level extracted from Apple mobility data and Family size as extracted from US Census. Since little is known about actual behavior, it was decided to use 3 possible behavior strategies as described in Table 2.

| Response | | | |
|---|---|---|---|
| Scheme | | | |
| # | Condition | Change in Interactions | Comments |
| 1 | No_Covid19 | (FamilySize − 1) + | Apple Mobility |
| | | Ceil(Max(0, (BaseInteractions- | interpolates level of |
| | | FamilySize + 1)*AppleMobility(State, | interactions beyond |
| | | Time))) | family size. |
| 1 | 10% random and | Max(FamilySize − | 10% infected people |
| | Covid19_Infected | 1, Floor(Uniform(0, | randomly reduce their |
| | | 1)*(Interactions*COVID19_Infected))) | number of interactions |
| | | | daily until family size is |
| | | | reached. |
| 2 | No_Covid19 | (FamilySize − 1) + | Apple mobility |
| | | Ceil(Max(0, (BaseInteractions- | interpolates level of |
| | | FamilySize + 1)*AppleMobility(State, | interactions beyond |
| | | Time))) | family size. |
| 2 | 20% random and | Max(FamilySize − | 20% infected people |
| | Covid19_Infected | 1, Floor(Uniform(0, | randomly reduce their |
| | | 1)*(Interactions*COVID19_Infected))) | number of interactions |
| | | | daily until family size is |
| | | | reached. |
| 3 | No_Covid19 | BaseInteractions | Healthy individuals do |
| | | | not change behavior |
| 3 | Covid19_Infected | FamilySize − 1 | Infected persons drop to |
| | | | interaction with family |
| | | | only. |

Behaviors are hard to assess, since here are many schemes of behavior that change from person to person and from location to location, yet the above possible behavior schemes represent extremes that may be reasonable under some circumstances. The last scheme represent an extreme person that does not change behavior due to a pandemic until getting infected. The first two response schemes represent a recrudesces in number of interactions during the pandemic which continue to decrease further during infection in different rates. Note that apple mobility data records requests to the web site and not actual mobility.

Note that recovered individuals go back to their normal behavior and the following formula is applied. Interactions=BaseInteractions. Also numbers of interactions for all alive individuals is summed to calculate TotalInteractions. Also Infectiousness is recalculated after number of interactions changes daily. Therefore the change in response scheme proportions in the populations changes interactions which effects transmission from two paths and makes the transmission probability a nested combination of the ensemble.

### Mortality Models

Mortality is a good example for a nested combination of the ensemble. Initially, the only mortality information located came from the CDC publication that contributed two models of mortality based on age-both presented at the table in the publication that provides lower and upper bounds-we will call these MortalityRate1(Age) and MortalityRate2(Age).

However, Mortality rate is not sufficient and there is a need to locate mortality time. An initial solution was to make different assumptions in form of mortality time models. The first assumption was extracted from a table in a publication of non survivor column-Time from illness onset to death or discharge, the days median (IQR) 18.5 (15.0-22.0) Since distribution information as not full, those were modeled as a Gaussian distribution: MortalityTime1=18.5+CappedGaussian3*(15.0−22.0)/0.674490/2 where CappedGaussian where CappedGaussian3 is a normal distribution that is capped at 3 STD to avoid extreme outliers. Another mortality time model was extracted from The Covid tracking project data by finding the first death per state since first diagnosis. The programmatically extracted distribution became: MortalityTime2=(13.345455+CappedGaussian2*6.287703) where CappedGaussian2 is a normal distribution that is capped at 2 STD. Note that those models generate two random numbers for each person and the combined mortality time for the ensemble becomes: MortalityTime=Σwᵢ MortalityTimeᵢ.

Once we have a probability of mortality and time of mortality it is possible to generate a random number and compare it to the mortality probability only at the designated mortality time that was also generated randomly. This was the first scheme of mortality.

The second scheme of mortality became possible once was presented in the Viral pandemics working group and a discussion about it in the integration subgroup mailing list led to replication of the model. This replicated models provide the probability of death of an individual per age group per day from infection we will call it MortalityPerDay (Age, TimeFromInfection).

Note that the formulation of the different type of mortality models make them hard to integrate as an ensemble. The construction solution was possible by assigning each individual a different mortality scheme randomly by proportions related to their influence weights: p₁, p₂ Such that p1 proportion of individuals have the probability of death of Eq (Time-InfectionTime, Floor (MortalityTime))*MortalityRate while p₂ proportion of individuals have the probability of MortalityPerDay (Age, TimeFromInfection). This is an example where the model combination is nested by proportion where one of the sub model combination is constructed by quantity and a formula. This complicates comprehension of the constructed model since there are multiple weights for multiple sub groups combined together. However, the model is still intelligible.

### Recovery

Recovery is difficult to define since there was little information on recovery and recovery competes with mortality so the transition probabilities should never rise above 1. Since recovery was not a point that is being measured in the validation, it was decided to simplify it and use the following formula:

Max(0,And(Eq(Infectiousness,0),Gr(Time−InfectionTime,MortalityTime),Ls(CombinedMortalityProb,1e−8))−CombinedMortalityProb)

An individual is considered recovered in the simulation if no longer infectious and time of death has passed or the probability of mortality is very low. The probability CombinedMortalityProb is subtracted to make sure that recovery probability plus mortality probability never rise above 1 or go below zero. Note that recovery is influenced by multiple model groups in the ensemble although there is only one equation.

### Simulation

The Reference Model simulation is relatively complex and demands computational resources. The simulation length is proportional to:

Size of each simulation batch that includes:

Number of individual simulated to represent the population of each state—In the largest simulation in this work there are 10,000 individuals per batch.

Time of simulation—in this simulation 68 days were simulated in each batch.

Number of populations simulated—in this work we execute the simulation for 51 US states and territories.

Number of repetitions of simulations—each simulation is different since it is based on random numbers. In some simulations patient zero may not even transmit the virus and in some the epidemic spreads quickly. We use the average of all those simulations. In the largest simulation there are 40 repetitions of each simulation.

Number of models in the ensemble. For M model coefficients/combinations there will be M+1 simulations. In this work we have 18 combinations of models in the ensemble.

Number of optimization iterations—in this work we attempt execute 10 optimization steps, yet convergence may occur before

Therefore the simulation has over 200K batches of simulation. Each batch has to go through simulation and report generation steps and a few more processes to aggregate the results and perform optimization. To perform such a simulation there is a need to use High Performance Computing (HPC). Due to importance of this work, multiple providers were gracious enough to contribute cluster computation time on two platforms: Rescale cloud credits were provided by Microsoft Azure and by Amazon AWS and the Midas Network provided their cluster. Moreover, many simulations were executed on a local 64 core server for many months. Overall there were 37 model versions executed since project start and over 100 simulations of different sizes. The reason for so many simulation was to eliminate errors and stabilize the model.

Typically a model version goes through these simulations: Formula simulation-simulations that just makes sure that all computational components work and there is no error in equations—this simulation works on a small simulation of 100 individuals per batch and only 3 repetitions and can be executed on a notebook for a few hours. its results are meaningless, it just makes sure that there is no grave error in equations and those interact well and can scale up. Small simulation—this simulation runs a model with 1,000 people per batch for a small number of repetitions or for a small number of states to give an idea of what results might be—the results are typically not stable due to small number of repetitions, yet it usually completes within hours or days on a 64 core machine and helps decide if to go back to modeling or to proceed to a larger simulation. Medium simulation—this simulation includes all states and either repeats a batches of 1000 individuals 100 times or repeats a simulation of 10,000 individuals for 10 times.

Note that a batch size of 10,000 increases the resolution of simulation since it allows modeling finer numbers of infected and deaths, while more repetitions reduces the statistical error associated with the Monte-Carlo error. This simulation typically has stable results and is already meaningful to extract some observations. Such a simulation takes many days on a local 64 core machine or hours on a cluster. Final simulation—this simulation is used to obtain final results for publication. It has many more repetitions of a population batch of 10,000 individuals—it was uses as much computing power as available to receive the best results possible by diminishing Monte-Carlo simulation error.

This scaling up of simulations allows improving quality of results while saving time and resources. Many times multiple simulations are executed in parallel knowing that one simulations will be stopped if a smaller one does not produce good results.

To improve simulation, the modified Gradient Descent (GD) optimization algorithm was enhanced to fit the concept of long computations between a small number of iterations. Before this publication the GD supported bounds and resealing of groups of model influences, in this version it also supports reduction of step size according to several strategies. The strategy used in this work is proportional reduction of step size if fitness score increases above a threshold—this may indicate overshoot and reduction of step size may help finer convergence.

The large number of versions and simulations is necessary to remove errors and test new models. Unlike regular programming, micro-simulation is less intuitive to humans and harder to debug. It is very easy to program errors that are hard to detect and currently there are no tools like a debugger for micro-simulation, so it is harder to fix and fixing a model takes much more time. Therefore, many versions and simulations were necessary for stabilization. Many errors were detected during this process and there is no full guarantee that the current version does not contain an error despite all efforts. However, the author believes that the current version was vetted enough and ready for publication since some phenomenon were observed enough times and did not change and since the model at its current version contains sufficient novel elements that warrant publication beyond the results.

### Results

The results presented here was executed on 32 nodes×36 cores=1152 cores total for almost 49 hours—this roughly means roughly 6.6 years or computation on a single CPU core. The results are presented as 3 main plots that the user can interact with: Population Plot—This plot shows the fitness score of each state population every 10 days as a circle. A viewer hovering with the mouse over the circle will see information about the population at that time including number of infections and deaths. The numbers are presented as model projection/observed numbers by the COVID tracking project. The numbers are scaled to cohort batch size during simulation, e.g. the number of deaths is from 10,000 individuals. The fitness score in this paper is: Norm2 (model death-observed death, (model infections-observed infections)/1000). Meaning that fitness is very close to death difference with slight influence from difference of infections. The reason for this fitness score is that COVID-19 death is much more accurate than infection numbers. Also note that outcome numbers compared are calculated using sum over last observation carried forward. Model Mixture Plot—This plot shows the influence of each model on the ensemble. Models from the same group that compete with each other are presented in the same color and their combined influence will be 1. Initially all models in a group have the same influence so in iteration 1—the plot shows many bars in the same height. When dragging the iteration slider and increasing the iteration, it is possible to see that some models gain influence while others lose it. In one case, the transmission model with 10% probability of transmission per encounter, the model is fully rejected by the model indicating that transmission probability is not that high considering all other assumptions. Note that the mortality models have 3 groups since we are combining models of different types in a nested manner. Convergence Plot—This plot shows the weighted average fitness for the US states and territories used for each iteration. The blue vertical line shows the current iteration, while the large yellow circle shows the fitness for the unperturbed simulation that is the base of the gradient descent. The small circles show the results for the perturbed simulations that help construct the gradient, each perturbing the result in one model coefficient that represents model influence. The small circles also represent sensitivity analysis—that we get for free while performing the optimization. The red horizontal lines represent the average fitness considering all the simulations. This plot clearly shows some models that are outliers in some iterations by being spread far away from the unperturbed solution.

### Discussion

An ensemble model allows us to explore our knowledge and assumptions about a topic while including many other assumptions. For example the DHS question from 26 May “What is the average infectious period during which individuals can transmit the disease?” can now be answered. Moreover, the answer is more elaborate and the average infectiousness model can be computed while taking into account multiple sources of data and models.

The answer may change if our set of assumptions change or if we ask the ensemble another question posed as a different fitness function or a different time period to compute the fitness on. However, without such an ensemble we would have had multiple assumptions and no good way to construct them together other than simple averaging-which does not allow comprehension of mechanisms that cause the disease. The Reference Model allows us to construct mechanistic models together in a way that is intelligible to humans. This technology is relatively new and requires much more exploration, yet it allows exploration that was not possible before.

Moreover, it is possible to easily extend this technique and allow including human interpretation similar to what was done for diabetes using the same technology. This way, it may be possible to answer questions like if the infection level in the population was overestimated or underestimated. is the infection in the population by combining computational models and human intuition and analysis.

### Example 2 Introduction

Computational Disease Modeling is a field where computational models attempt to predict outcomes for a population or an individual by using computer models. Those models many times are expressed as risk equations that attempt to predict the probability of an outcome in a patient with specific characteristics For example what is the probability of a patient experiencing stroke in 10 years given their age, blood pressure and other parameters. Those risk equations are typically developed by a modeling group that has access to longitudinal data of patient data.

Typically patient data in the medical world is highly restricted and is rarely shared with other groups, so publishing the risk equation/model is one way of sharing knowledge that does not compromise the restricted data. However, combining this knowledge was very limited for many years. Assembly attempts by some groups included assembling their own equations to models that predict multiple outcomes and others assembled equations from multiple sources into one model. Yet at this earlier time, global assembly of information was not possible.

A lot of progress was done in the diabetes modeling community and modelers started comparing their model in the Mount Hood challenge where multiple modeling groups would meet to compare and contrast their models. However, the models constructed by multiple teams were different and results varied across multiple groups when validation challenges were attempted. In validation challenges, baseline population statistics were given and modeling teams were competing in how close they can predict the outcomes for that populations. Populations typically represented clinical studies with a few executions, so summary data was publicly available. Despite the availability of data, the predictions provided by multiple teams varied and were not accurate. Moreover, each time a modeling challenge was introduced, there was no continuity to previous challenges and validation against populations from previous challenges was not required in a newer challenge.

Although attempts were made to standardize input data for challenges, the process was a human intensive process focused on the modeling teams making assumptions and interpreting ambiguous data rather than an organized procedural process that can be automated.

The inability of the diabetes modeling groups to replicate known outcomes and the variety of models inspired the author to take a new approach that will merge information from multiple them against multiple sources in an automated manner. The Reference model was the solution.

### The Reference Model for Disease Progression

The Reference Model started with the idea to automate the Mount Hood challenge. Instead of multiple groups of humans meeting once every other year and preparing for a few months for one challenge, a machine can receive all models and run them on the same standardized inputs. This can happen continuously and also allow accumulation of knowledge in one place so that multiple challenges can be stored together. Yet once the problem was formulated for a computer, it opened many more possibilities for accumulating knowledge as will be described later. Yet we are ahead of ourselves and should start with the first model version.

The Reference Model was created in 2012 as an automated mini replica of the Mount Hood Challenge aimed at diabetic populations. The model included 3 processes coronary heart disease, stroke, and competing mortality. This structure of the model was relatively simple. The arrows in the model diagram represent transitions between disease states. During simulation a random number is picked for each active state and it is compared to the risk equation that represents a threshold for transition. This way the model decides if an individual moves to a different state or stays in the same state for that time step. This is repeated for each individual in the population. At the end of simulation the model outcomes are compared to known population outcomes to figure out how good the model is, we will call this number fitness.

Despite its simplicity, the model allowed complexity that was not possible with the human based challenges, it allowed assembling a model using different risk equations. Each transition probability could be represented by more than one risk equation. The Reference Model was therefore not one single model, it was an ensemble model that is composed of many models. However, initially the full potential of the model was not realized since the different models were made to compete-very similar to what was done at the Mount Hood Diabetes challenge. Each time a simulation executed; a different equation was chosen for each transition probability. For example Equation A would be chosen for the probability for Myocardial Infarction (MI) and Equation E was chosen as the probability of Stroke-denoted by the combined model AE. We could contract multiple such models: AE, AF, AG, AH, BE, Bf, BG, BH, CE, CF, CG, CH, DE, DF, DG, DH and this number would grow up exponentially and therefore High Performance Computing (HPC) was required to run all those models and figure out which one represents best the phenomena observed in the population. And this was executed for multiple populations to figure out the model that behaves best for all populations. This approach was competitive and although it allowed accumulating more knowledge than the human challenges that lacked consistency by removing previous challenges, it did not reach full modeling potential.

The full potential was realized after the number of models and populations grew, it was then necessary to switch to a much better approach that utilized the full potential of the ensemble model-a cooperative approach. The key observation was that no one model is perfect and all models should be treated as assumptions rather than absolute truths and we wish to merge assumptions together so those will cooperate. In this cooperative approach, all risk equations contributed to a combined risk according to their influence. For example, for the MI probability equations was assigned a weight and the combined probability for a transition was w1A+w2B+w3C+w4D where the coefficients w1,w2,w3,w4 are scalar weights that represent the influence of a certain equation. The Reference Model then represented an infinite number of models that represent disease progression based on risk equations as basis functions. The modeling space then became a continuous function that can be optimized using mathematical optimization techniques that are very similar to those used in training neural networks (Barhak, 2016). The solver was named as: “assumption engine” since it figures out which assumptions work better together considering the data and query. This cooperative approach allowed creating models that behave better than any of the original risk equations alone. Moreover, it allows testing assumptions that are not continuous in nature.

Information accumulation went beyond multiple models being integrated into one ensemble model. Much important information is provided by population data that was also incorporated. The Reference Model started with validating against a few past populations from the Mount Hood challenge and the literature. This number increased with additional challenges. Yet unlike the human challenges that did not retain memory from previous validations, the ensemble model retained those and this data was accumulated rather than forgotten. The Reference Model uses population data that was publicly composed of summary statistics rather than restricted individual data that is typically not released. The model needed to simulate populations that matched the demographic of those population cohorts. This was done by sophisticated population generation driven by the MIcro Simulation Tool (MIST) (Barhak, 2013) that served as the computational engine behind the model. Since population generation was a Monte Carlo random process, there was a need to improve accuracy to better match population statistics. This was accomplished using Evolutionary Computation algorithms (Barhak & Garrett, 2014). However, when the model grew, the amount of code that was required became unreasonable and object oriented population generation code was introduced to allow efficient and compact population generation (Barhak, 2015).

Yet even with efficient ways of recreating populations, the process was slow—it took roughly a week of work to recreate one population from a publication and much of this work relied on copying numbers from published papers and writing generation code. This was remedied when an interface was created for ClinicalTrials.Gov that reduced the time required to add a population to a few hours per population, while eliminating human error.

ClinicalTrials.Gov is the registry where clinical studies report their structure and results. This database growth is driven by U.S. law and already holds over 300,000 clinical studies with over 41K clinical studies with results. Results data that was previously published without uniform format in scientific journals is now entered into a database. An interface was created that allows the modeler to use extracted data and semi-automatically create populations that can be simulated by the ensemble model. This interface caused a dramatic increase in the amount of knowledge held by the model. The Reference Model then became the most validated diabetes cardiovascular disease (CVD) model known worldwide, bypassing the previous champion—the Archimedes model (Eddy & Schlessinger, 2003). Today, there is no other known CVD diabetes model that accumulates information from so many sources with validation.

With so much information, it was then possible to visualize our computational knowledge gap. This gap shows how the most fitting model assembled from the base equations fits all clinical studies. This was presented using interactive techniques based on Python visualization libraries (Bokeh, Online), (Holo Viz, Online).

With so much information assembled, it was possible to analyze data in ways not possible before. For example the rate of improvement of treatment in CVD diabetic death could be assessed, so a similar idea for Moore's law could be defined. the model discovered that diabetic CVD death probability decreased roughly by half every 5 years as calculated using 3 decades of models and populations (Barhak, 2017). Life tables were published using two scenarios: 1) using improvement rate into account, 2) not correcting for treatment improvement rate. This was just one example of what is possible when information from multiple sources is centralized in one ensemble model.

However, despite all the progress made, information arriving from multiple sources is still prone to human error despite capabilities of detecting wrong equations. Even strict testing was shown to bypass a few errors each year. For example, the results in this paper correct a row shift and a mismatch in a result matrix that was introduces by human errors in the two last published versions However, more automation and accumulation of knowledge will eventually diminish a possible error to be negligible and hence the need to go away from human focused modeling to automated modeling. For example, the erroneous outcome entry in the last publication (Barhak, 2020) is only one from 120 outcomes entries and therefore if its influence is not strong when comparing results and can be considered negligible. Moreover, one equation know to be erroneous is rejected by the model on the first iteration, thus demonstrating how accumulated knowledge effectively reduces error.

However, even if the process becomes highly automated, humans still need to be involved in the modeling process. Humans, just like models, have different opinions and many times there is no easy way to measure the accuracy of those opinions. Since humans need to drive the modeling process, instead of the human being concerned with performing repetitive tasks, humans should be focused on looking at data and results. In this paper we introduce one way of doing this by including human interpretation to deal with ambiguous or fuzzy data while employing machine learning to figure out the best fitness when considering interpretation by a team of experts.

### Handling Human Interpretation

When transforming medical data into a model there are many human considerations taken. Many of those are not computational in nature and relate more to understanding texts. Despite advances in Natural Language Processing (NLP) machines still cannot perform human language interpretation properly and computational model creation based on such data is even a harder task. However, for a computational model that validates predictions to outcomes, it is possible to pose the problem in a way a machine can comprehend.

Outcomes of a clinical study are typically counts of a certain observed phenomenon, for example a stroke. However, a stroke can be defined in many ways and therefore different trials may report the same outcome differently. Sometimes the definition of an outcome is made using International Statistical Classification of Diseases (ICD) codes.

However, even when well defined in one ICD version, the definition may change in another ICD version. For example in (Clarke et. al. 2004) ICD 9 Stroke is defined by as (ICD-9 codes >430-<434.9, or 436) However, when translating to ICD 10 codes, the list closely translated to I60.9, I61.9, I62.1, I62.00, I62.9, I65.1, I63.22, I65.29, I63.139, I63.239, I65.09, I63.019, I63.119, I63.219, I66.09, I66.19, I66.29, I63.30, I66.9, I63.40, I66.9, I67.89. Only looking at the first code of ICD9-430 the definition is “Subarachnoid hemorrhage” while the ICD 10 160.9 equivalent is defined as: “Nontraumatic subarachnoid hemorrhage, unspecified” these small changes in definition eventually cause confusion for a machine when the word stroke appears in a published report. Although a human will be able to explain what a stroke means, for a computer a different definition of the words that describe stroke or a different code list will be hard to decipher.

This problem aggravates further since in tables that describe clinical study results, the ICD codes that define a specific outcome are not specified directly and although many times those can be found after an exhaustive human search in the trial protocol or in another location in a related publication, many times there are differences in reporting outcomes between trials. The problem aggravates even further in composite outcomes such as cardiovascular disease (CVD) that include many other outcomes including MI and stroke. The definitions of outcomes sometimes even differs within the same clinical study that reports the same outcome using different definitions.

For example the RECORD clinical study (ClinicalTrials.gov-NCT00379769, Online) reports the same outcome twice using two different criteria: 1) “Independent Re-adjudication (IR) Outcome: Number of Participants With a First Occurrence of a Major Adverse Cardiovascular Event (MACE) Defined as CV (or Unknown) Death, Non-fatal MI, and Non-fatal Stroke Based on Original RECORD Endpoint Definitions” 2) “Independent Re-adjudication Outcome: Number of Participants With a First Occurrence of a Major Adverse Cardiovascular Event (MACE) Defined as CV (or Unknown) Death, Non-fatal MI, and Non-fatal Stroke Based on Contemporary Endpoint Definitions” Although this trial has properly reported the outcomes using multiple interpretations, it is unclear how to compare those outcomes to a different trial and how to validate those against simulated model outcomes, especially when an ensemble model is considered—the description is not traceable back to quantifiable definitions and therefore hard to a machine.

Similar definition changes are not uncommon, the definitions in medicine change constantly even outside cardiovascular disease. For example the definition of sepsis was changed numerous times in a few decades as seen in (Gary et. al., 2016), (Wentowski et. al., 2018). And since the model accumulated clinical information spanning over several decades, there is a necessity to add human interpretation to outcomes being used for validation.

However, note that humans may not always understand the data the same way, and human interpretation of the same outcome may differ from one expert to another. The example of the RECORD study (ClinicalTrials.gov-NCT00379769, Online) discussed earlier shows how the same outcomes are interpreted differently and numbers differ. So we wish to be able to add human interpretation of outcomes from multiple experts that will evaluate possible ambiguous information.

In the past, the Delphi method was used to assemble information from multiple experts. One example of a derivative of the method was used for mental health modeling (Leff et. al., 2009). However, those techniques are human based and require human feedback and reiteration which is time consuming. We want a technique that takes human inputs and allows merging it efficiently with the power of machines to dates the assumptions that experts make.

### Mathematically Handling Human Interpretation

Human interpretation can potentially be added to any aspect of modeling, yet it was initially applied only to outcome interpretation. Consider the following notations:
fi—simulation result—this is the number the model generates after Monte Carlo simulation.
F—expected target outcomes—these are the numbers that appeared at the clinical study results—
our ground truth Hi (T)—Human interpretation of T by expert
i—representing what the expert thinks the ground truth should be
D—difference between ground truth and simulated results—this is the fitness/error we wish to me minimal.
wi—the weight we assign to expert i interpretation—it represents how much we believe that expert

The basic idea is to find the best balance of experts that will increase the prediction accuracy of the simulation. The Reference Model uses a fitness engine that calculates the difference between simulated results and expected outcomes and attempts to optimize it. Without Human interpretation, this would be defined as:

$$D = T - R \to MIN$$

However, when we introduce human interpretation, this difference becomes a weighted sum considering all experts:

$$D = Σ wiHi ( T ) - R \to min subject to : Σ wi = 1 and wi \geq = 0$$

The constraints make sure that the combined weighted interpretation of all experts is within the convex hull of all the interpretations given and that no interpretation given by an expert is considered as false—at worse case the interpretation is incorrect if w,=0 In simpler words it means that the minimum and maximum after accounting for all expert interpretations will be bound by the largest and smallest outcome interpretation of the experts.

Also note that the assumption engine already includes a very similar formulation where w, also decides the level of influence for a certain model equation as described before when assembling the ensemble model: w₁A+w₂B+w₃C+w₄D. In fact the interpretation of the expert can be considered part of the modeling assumptions that require optimization. The only difference is that to calculate the fitness D for interpretations there is no need to recalculate the results fi-which involves the entire simulation that involves validation of the population against the model-which is time consuming and typically takes about 16 hours on a 64 core machine to account for all variations and populations. Instead, we can quickly calculate all variations of interpretations very quickly without the need to recalculate fi. And since the assumption engine already uses gradient descent optimization to improve wi, for model components (Barhak, 2016), we just add an extension of wi, related to human interpretations to the solution vector and use the same solver rather than decoupling the human interpretation handling from the model assumptions handling. Here is proof that this decoupling is possible.

Lets call the Difference between ground truth and human interpretation of expert i as Di=wi(Hi(T)−R)

We will define the combined difference instead as: D=ΣDᵢ=Σwᵢ (Hᵢ (T)−R))=Σ(wᵢHᵢ(T)−wᵢR)=Σ(wᵢH(T))−Σ(wᵢR)=Σ(wᵢH(T))−R*Σ(wᵢ)

Since Σ(wi)=1 we get again: D=Σ(wᵢHᵢ (T))−R, which means that we can decouple the simulation from interpretation for the sake of determining interpretation weights of experts for optimization purposes. So when running the code we use the D=ΣDᵢ, formulation to deduce the combined interpretation difference.

Yet this description is still somewhat simplified compared to actual code that implements the simulations since each outcome appears in some populations. The actual way that experts interpret outcomes is by looking at the outcome description of a specific trial and expert i assigns a scalar number z, associated with outcome for a specific trial j. this number is used to adjust the ground truth Tⱼ for all cohorts of trial j so that Hᵢ (Tⱼ)=zᵢⱼ*Tⱼ. If zᵢⱼ=1 it means that the expert believes that the reported outcomes match the model definition of the same outcome. If zᵢⱼ<1 it means that the outcome defined by the study over-counts incidence compared to how the model views the definitions. if zᵢⱼ>1 then the study results in the publication does not include some outcomes defined by the model and the under-counted observed outcome should be increased to match the model definition. Also note that the model definition includes multiple merged models with different weights. Since all weights are optimized, the most fitting balance of all interpretations and assumptions is created-optimally mixing the model and expert definitions.

### Implementation

The Reference Model code was modified to incorporate human interpretation optimization as described before. As explained earlier, the code change could be merged with existing optimization code. Therefore, a lot of effort was put into handling the data. However implementation included multiple other changes. One minor change added warning code to isolate an issue with an equation that was previously marked as wrong by the assumption engine.

The major change was that all outcomes that were reported by all studies entered into the system were revisited. Those study outcomes were previously matched with model definitions of outcomes using free text that explains the modeling assumption and as a table matching the outcome to ICD codes, this was done for MI, Stroke, CVD and mortality and their combinations. Much effort was put previously in documenting the modeling assumptions regarding outcome definitions, yet this was only a documentation file. In the new version this documentation was adapted to a matrix of human assigned values that can be incorporated into computation. Each row in the matrix of values contained a single outcome extracted from a certain study including human explanation. There were many columns in that matrix, most of which contained documentation. A few numeric matrix columns were added to contain numeric human interpretations. Ideally each column should have represented a different expert opinion on how well the study outcome matches the model definition as a positive number around 1. Those values correspond to the zᵢⱼ values that go into computation.

In this publication, only the author wrote all interpretations while trying to imitate 6 experts with different opinions both conservative and liberal-we mark them as 1-6 in Table 3 below, each time making other assumptions trying to simulate conservative experts that stick to the textual definitions and emphasize the difference by assigning numbers farther than 1 in a direction that fits their “assumed personality” More liberal experts may accept differences in text more easily and report numbers closer to 1. Note however, that death was considered absolute outcome that all experts gave the interpretation of 1. The first interpretation in the interpretations columns was full of 1 values indicating that model outcome matches study outcome. Note that Table 3 provides only a small glimpse into the interpretations used for a small number of the 120 outcomes used in the simulation
just to illustrate the procedure.

TABLE 1
Small subset of the interpretation data

| | | Expert Interpretations | | | | | | | |
|---|---|---|---|---|---|---|---|---|---|
| Study | Outcome | 1 | 2 | 3 | 4 | 5 | 6 | Reference | Comment |
| UKPDS33 | Death | 1 | 1 | 1 | 1 | 1 | 1 | (UKPDS,1998) | All deaths counted |
| ADDITION | MI | 1 | 1 | 1.2 | 0.8 | 1.2 | 0.8 | (Griffin et. al., | Exact detailed definition is not |
| | | | | | | | | 2011) | available in the paper, and since it |
| | | | | | | | | | is a multi national trial, it is |
| | | | | | | | | | assumed that there is some |
| | | | | | | | | | variability beyond MI + Stroke |
| ADDITION | Death | 1 | 1 | 1 | 1 | 1 | 1 | (Griffin et. al., | Death is absolute |
| | | | | | | | | 2011) | |
| RHCORD | NR | 1 | 1 | 1 | 1 | 1.05 | 0.95 | (ClinicalTrials.gov | Word description is very specific |
| | | | | | | | | NCT00379769, | and short with little room for |
| | | | | | | | | Online) | interpretation of MI |
| PROACTIVE | TO + | 1 | 0.6 | 0.7 | 0.5 | 0.8 | 0.4 | (ClinicalTrials.Gov | Includes many more elements |
| | Stroke + | | | | | | | NCT02678676, | including amputation and |
| | Any | | | | | | | Online) | procedures-needs a reduction for |
| | Death | | | | | | | | sure |

Note that the interpretations here were given by one person “impersonating” several opinions. Yet after computation, a merged interpretation is created by weighting all those interpretations together in a way that best matches all the other data and assumptions added to the system with regards to the query used. The spread in expert interpretations also can be used do define possible bounds for the ground truth value—is it quite possible for an expert to have several opinions on what is possible in case variability is large. The assumption engine will find the best fit.

### Results

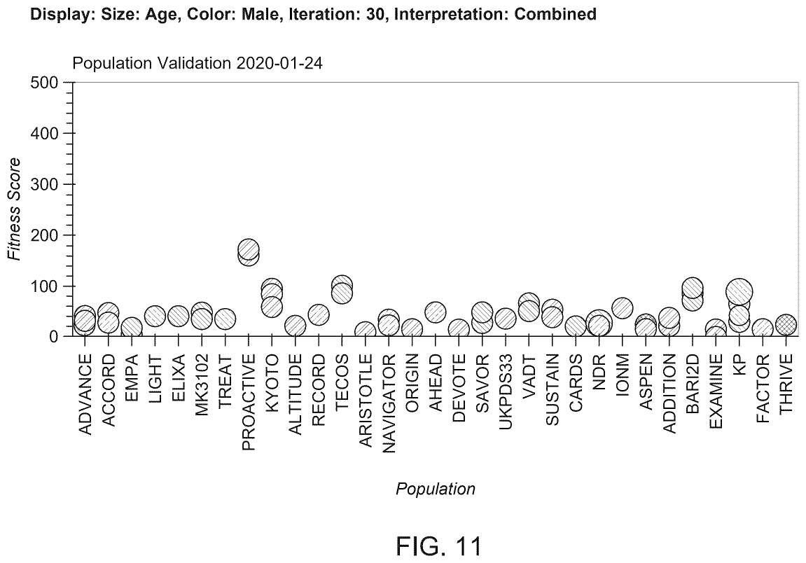

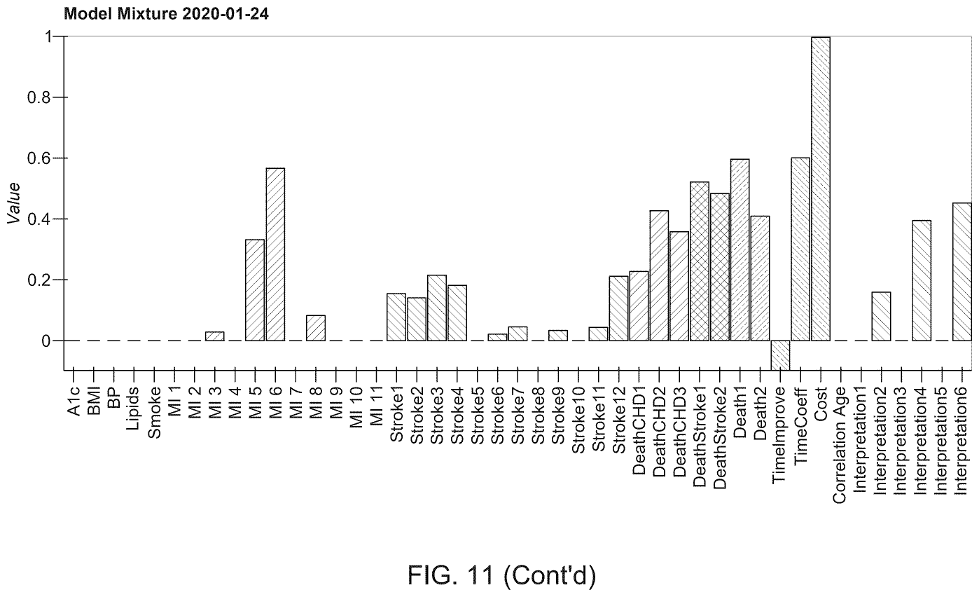

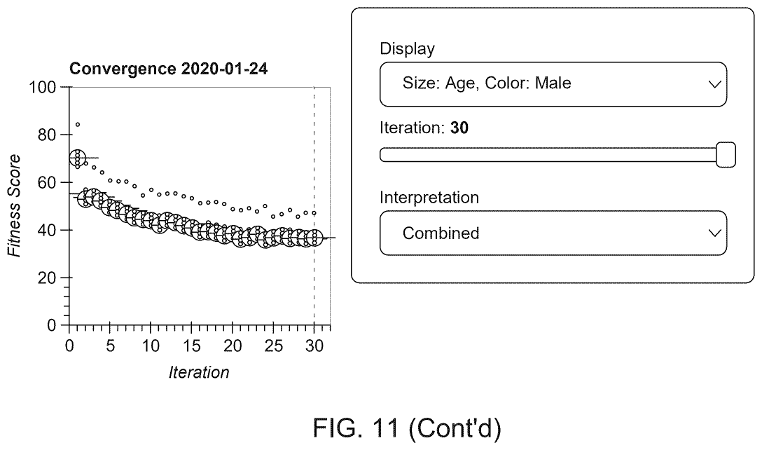

Simulation was conducted on a 64 core machine for 3 weeks. 30 optimization iterations were calculated to determine the most fitting model combination and the most fitting expert interpretation. When simulation started we already expected that one of the implemented risk equations that was shown to be misbehaving in the past would be eliminated by assumption engine. From past results it was known that the population we called PROACTIVE (ClinicalTrials.Gov NCT02678676, Online), since it was based on a previous trial enrollment with this acronym, was a severe outlier as can be see here (Barhak, 2019). So we expected that Expert 1 interpretation will be rejected by the assumption engine. Recall that expert interpretation 1 simulates an expert that believes that the model outcomes are defined the same as the study outcomes-looking at the clinical study definition of the outcome, we know this is not reasonable and in fact this may have been better if this trial was excluded from validation due to incompatibility. However, in this work it serves a purpose of showing how human interpretation can help explain things. The results generated do support our prior knowledge and MI equation 11 and expert interpretation 1 weights are both zero at the end of simulation as can be seen in FIG. 11.

The Reference Model Visualization was enhanced once more this year to use the most advanced HoloViz python technology to visualize the results interactively. Those interactive visualization allow hovering with the mouse over plot elements to get more information. To supplement this paper, some iterative visualization are available interactively. FIG. 11 statically as one snapshot.

FIG. 11 shows 3 plots: the top left plot represents clinical studies cohorts and their fitness. Each circle is a clinical study and its color/size represent Age and proportion of Male and their height represents the fitness of model prediction to the observed outcomes of the clinical study cohort. Fitness may include multiple outcomes associated with the study that are merged into one number, for the sake of simplicity think about it as simulation error measure for that cohort, defined by the query posed to the model. So a higher circle on the vertical axis, means that that cohort results cannot be explained well compared to a cohort that is represented by a lower circle. Ideally we want all circles to be as close to zero as possible, meaning that our ensemble model is very good. However, this is not realistic, since even observed clinical study results have statistical variability. However, this plot is useful since it shows us what we can explain well computationally. In the future addressing issues that cause some cohorts to be predicted poorly, may improve fitness. So this result give a reference for comparison of our cumulative computational knowledge. The more information that can be absorbed Into the model the better we can see how well computers can explain and predict a phenomenon. The Reference Model is than important as a map for exploration of the ability of machines to comprehend medical knowledge.

The bottom plot in FIG. 11 represents the weights that construct the best model. Each bar is associated with a certain equation, while equations that represent the same transitions have the same color. The last group of bars colored cyan is associated with the interpretations. It is clear that there is no bar for MI equation 11 and no bar for expert interpretation 1, meaning that those assumptions have been rejected by the assumption engine as not contributing to the most fitting model.

The Top right plot represents the convergence of the model in each simulation iteration. The overall fitness score, that is a weighted average of cohort fitness scores, is shown as big circles. The fitness of gradient components is shown as smaller circles. It can be seen how the simulation converges and stays more or less steady after 30 iterations. Since the simulation is Monte-Carlo based it is expected to see some fluctuations, yet the results show clear convergence. If we look at the last combined fitness score of 36 out of 1000 and trying to best interpret the math, we can very loosely say that according to all the knowledge accumulated to date, and while making many simplification in result interpretations, we can predict outcomes on average with fitness of 3.6%. This is our current cumulative gap of computational knowledge and an improvement of 1.4% over the result on 2019 (Barhak, 2019).

The Reference Model in about 8 years of development accumulated more computational knowledge than ever was reported to be accumulated by any diabetes CVD model. Not only it can absorb other models, assumptions, populations, it can now also include human interpretation. The ensemble model now allows automation of significant portions of the modeling process, processes that were once, and even today, done manually.

The Reference Model rise in capabilities by automation should also be contrasted against the decline in human modeling capabilities as reflected by the Mount Hood Diabetes challenge group. The Reference Model was initially created to imitate and improve some processes happening in validation challenges in 2010. In 2012, 2014 the human modeling groups participating, did not validate their results against previous year results while the ensemble model did validate against all previous populations-8 in 2014. The Mount Hood challenge in 2014 only validated against one population and in 2016 no more populations were introduced for validation, while the ensemble model grew in its validation capabilities in these years while adding those to previous populations and reaching 9 population in 2016 and today stands on 30. The decline of the human modeling paradigm was very clear in 2016 Mount Hood Diabetes Challenge where human groups, including the author, were asked to recreate previous models without success by any team. This alone proves that humans should not be performing repetitive modeling tasks that are better done by machines. However, human decline has reached a new low when some participants in the challenge decided to republish the 2016 challenge results while omitting results-humans can decide to do this, while machines do not remove data willingly. The Reference Model results were removed while it was the only model that has reproducibility tests build within it-see reproducibility section below. During the challenge and afterwards during the summary process the author has called multiple times for publication for code for reproducibility and the idea was not adopted by the human led group.

This decline in human modeling approach compared against rise in automation capabilities and accumulation of knowledge by machines happens in other aspects of our lives like driver-less car technologies that are slowly developing. However, despite machine automation rise, humans still have value and their opinions and needs should be collected by machines in proper manner. The machine automate tasks well, while humans should have a good interface to guide the machines to reach desired goals. The Reference Model now has proper interfaces for humans that fulfill the following roles: 1) Modelers can add new models/assumptions to our knowledge, 2) Data experts/Bio statisticians can archive clinical study data to be validated against 3) Medical experts can interpret clinical study definitions.

Using those interfaces and further improving automation and gathering of data, it would be possible to improve our model prediction accuracy in the future. At some point in time, machine foecast accuracy should become comparable to the average medical expert prediction—this phenomenon is already reported for other machine automated tasks. When this point is reached and validated, it may be possible to discuss government approval of deploying such technologies. In fact the government is already preparing towards such scenarios (FDA-SaMD, Online). Some prediction on when this machine takeover may happen can be found in (Barhak & Schertz, 2019). The good news are that deployment of machine based technologies is easy and fast compared to deployment of traditional medical knowledge that is accomplished by long cycles of training humans, recruitment, knowledge exchange, and retirement, that take years. Software deployment, even considering hurdles is much faster. So the time from policy approval to deployment is relatively fast, and human adoption will not be hard for technologies that proved themselves if human concerns are addressed.

Therefore the current effort should be in improving the ability of machines to predict and accumulate knowledge. The Reference Model is only one tool in this struggle and it shows that our cumulative computational capability still needs improvement. However, other technologies that help in accumulation of data and its standardization like (ClinicalUnitMapping.COM, Online) are already under development and will allow improving the knowledge accumulation pipeline.

The following additional embodiments and implementations expand upon the systems and methods previously described. These enhancements provide further capabilities and clarifications to the analysis and verification of models derived from clinical studies data. Data extracted from clinical studies databases often contain diverse units of measure that require standardization prior to processing. The implementations described herein provide an enhanced system for mapping and standardizing units of measure with context awareness and standard-specific transformations.

Clinical studies involve extensive data collection and analysis, often resulting in datasets with diverse units of measure that vary across data sources, geographic regions, and reporting standards. This diversity of units creates significant challenges when aggregating data from multiple sources for comprehensive analysis or when building disease models that rely on standardized inputs.

Traditional approaches to unit conversion rely on fixed mapping tables or simple rule-based systems that fail to account for contextual information that might clarify ambiguous unit notations. These systems also struggle with variations in how units are textually represented and lack the ability to standardize units according to specific interpreter standards that may be required by different stakeholders or regulatory frameworks.

Furthermore, existing systems typically require complete information about units to perform conversions, with limited ability to work with partial information. This creates obstacles when processing historical or incomplete clinical data where full unit specifications might be absent.

For clinical studies data in particular, the standardization of units is critical to enabling accurate disease modeling and data aggregation. Current methods for processing units of measure from clinical studies lack the sophistication necessary to handle the complexity and variability inherent in real-world clinical data. There remains a need for intelligent systems that can standardize units of measure with greater accuracy, flexibility, and context-awareness than conventional approaches.

The present disclosure describes systems and methods for AI-assisted unit of measure standardization with context awareness and standard-specific transformations. The described implementations provide an enhanced system for mapping and standardizing units of measure from clinical studies and other data sources.

In one implementation, a unit mapping system utilizes artificial intelligence to transform non-standardized units into standardized formats based on one or more of: unit text, unit context, and interpreter standard selection. The system includes a database containing standardized units, unit text variations, unit context information, and interpreter standards; a neural network that processes inputs to generate suggested standardized unit mappings; a nearest unit search component that matches neural network outputs to permitted standardized units; and an output component that provides the standardized unit.

The neural network processes one or more optional inputs: unit text (the textual representation of a unit of measure), unit context (the contextual usage information about the unit), and interpreter (the standard to which the unit should be mapped). The system is designed to function effectively even when only partial information is available, making it robust for real-world clinical data processing scenarios.

In some implementations, the AI model is implemented as a transformer neural network or similar architecture, trained on datasets containing unit text, context, interpreter standard, and correct mapping outcomes. The system can be extended with a unit conversion component that combines standardized units with appropriate conversion formulas to transform values from one unit to another.

The systems and methods described herein may be implemented as standalone services accessible via web interfaces or APIs, or as components integrated into broader disease modeling systems. When integrated with disease modeling systems, the unit standardization capabilities significantly enhance data processing by enabling consistent interpretation of heterogeneous unit representations from disparate sources.

The present disclosure is directed to AI-assisted unit of measure standardization with context awareness and standard-specific transformations. The implementations described herein provide an enhanced system for mapping and standardizing units of measure from clinical studies and other data sources.

### Unit Mapping System Architecture

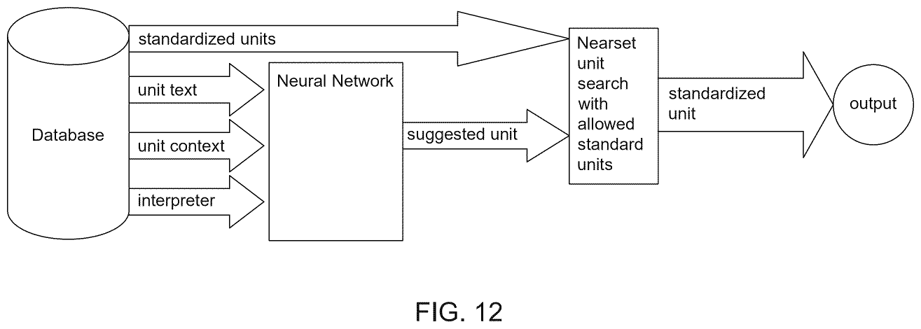

FIG. 12 illustrates a schematic diagram of an exemplary AI-based unit mapping system. The unit mapping system includes several key components. The unit mapping system has a database containing standardized units, unit text variations, unit context information, and interpreter standards. The unit mapping system has: a neural network that processes inputs to generate suggested standardized unit mappings; a nearest unit search component that matches neural network outputs to permitted standardized units; and an output component that provides the standardized unit.

The database component stores information necessary for the standardization process, including: a comprehensive list of standardized units in various measurement systems; variations in how units may be textually represented (e.g., “kg/m\hat{ }2”, “kg/m2”, “kilograms per square meter”); contextual information that helps disambiguate units in different usage scenarios; and standards specifications (interpreters) that define how units should be standardized for different reporting requirements or regulatory frameworks

The neural network component serves as the core intelligence of the system, processing one or more optional inputs: Unit text (the textual representation of a unit of measure as it appears in source data); Unit context (the contextual usage information about the unit, such as what it measures (e.g., “Starting body mass index”)); and Interpreter (the standard to which the unit should be mapped (e.g., a specific regulatory reporting standard)).

The neural network may be implemented as a transformer neural network or similar architecture, optionally with modifications to facilitate improved mapping to standardized units. The system is trained on datasets containing examples of unit text, context, interpreter standard, and correct mapping outcomes. All the inputs to the neural network are optional. This enables the system to produce the best standardized unit according to whatever combination of inputs is provided. For example, the system can infer a standardized unit given only the unit text and interpreter, without requiring unit context. Similarly, given only unit context, the system will attempt to determine the best matching standardized unit based on that limited information.

The nearest unit search component compares the output of the neural network to the permitted standardized units in the database. This ensures that the final output conforms to accepted standards rather than generating novel unit representations. The component may employ semantic similarity measures, exact matching, or other comparison techniques depending on implementation requirements.

The output component delivers the standardized unit in the format required by downstream processes. This may include machine-readable formats for automated processing or human-readable formats for display in user interfaces.

### Neural Network Implementation

The AI component of the unit mapping system may be implemented using various neural network architectures. In one implementation, a transformer neural network architecture is employed due to its effectiveness in processing textual information and capturing contextual relationships.

The transformer network is trained on datasets that contain examples of: Input unit text (e.g., “kg/m\hat{ }2”); Unit context information (e.g., “Starting body mass index”); Interpreter standard (e.g., “CDISC” or other reporting standard); and Expected standard unit output (e.g., “kg/m2” formatted according to the specified standard).

During training, the network learns to recognize patterns in how units are represented textually and how context influences the appropriate standardization. It also learns the specific requirements of different interpreter standards, enabling it to produce outputs conforming to the specified standard. The system has an AI model behind it that was trained on datasets that consist of a table that looks like the following Table 4:

TABLE 4

| unit of measure | context | interpreter | mapping |
|---|---|---|---|
| “kg/m\hat{ }2” | | | kg/m2 |
| “kg/m\hat{ }2” | | CDISC | kg/m2 |
| “kg/m\hat{ }2” | | IEEE | kg m-2 |
| | Starting body mass | | kg/m2 |
| | index (starting BMI) | | |
| | Starting body mass | CDISC | kg/m2 |
| | index (starting BMI) | | |
| | Starting body mass | IEEE | kg m-2 |
| | index (starting BMI) | | |
| “kg/m\hat{ }2” | Starting body mass | | kg/m2 |
| | index (starting BMI) | | |
| “kg/m\hat{ }2” | Starting body mass | CDISC | kg/m2 |
| | index (starting BMI) | | |
| “kg/m\hat{ }2” | Starting body mass | IEEE | kg m-2 |
| | index (starting BMI) | | |
| day | | | day |
| day | | CDISC | day |
| day | | IEEE | d |

The network is designed to handle incomplete information, making it robust in real-world scenarios where not all information may be available. This is achieved through training techniques such as masking inputs during training, forcing the network to learn to make predictions with partial information.

Beyond recognizing units of measure, the network may also be trained to distinguish units from non-unit text such as names, common words, or other text that might appear in clinical data. This capability helps prevent false positives when processing mixed content.

### Advanced Unit Conversion System

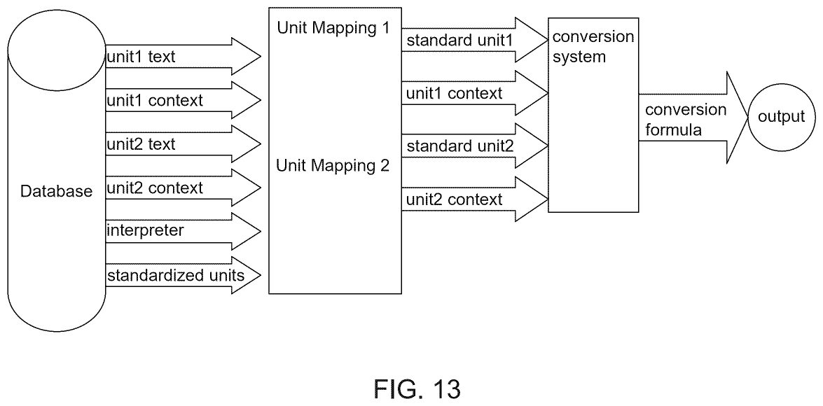

FIG. 13 illustrates a schematic diagram of an exemplary unit conversion system that builds upon the unit mapping system. The unit conversion system combines one or more unit mapping components that standardize input units, a conversion system that determines appropriate conversion formulas between standardized units, and an output component that applies the conversion and returns the result.

The conversion system component maintains knowledge of mathematical relationships between different units of measure. After standardization, it applies the appropriate conversion formulas to transform values from one unit to another. For example, after standardizing “kg/m\hat{ }2” and “lb/in\hat{ }2” to their canonical forms, the conversion system would apply the appropriate mathematical formula to convert values between these units.

In some implementations, the conversion system may also take into account context-specific conversion factors. For example, the conversion from mg/dL to mmol/L uses different conversion factors depending on whether the measurement is for cholesterol, glucose, or other substances. The system can use the context information to select the appropriate conversion factor.

The unit conversion process typically follows these steps: Standardize the source unit using the unit mapping system; Standardize the target unit using the unit mapping system; Determine the appropriate conversion formula between the standardized units; Apply the conversion formula to the input value; Return the converted value

The standardization step enables the system to identify the correct conversion formula. Without proper standardization, direct conversion between non-standardized units would be nearly impossible due to the vast number of possible textual representations and ambiguities.

Multi-Model Systems with Reasoning Components.

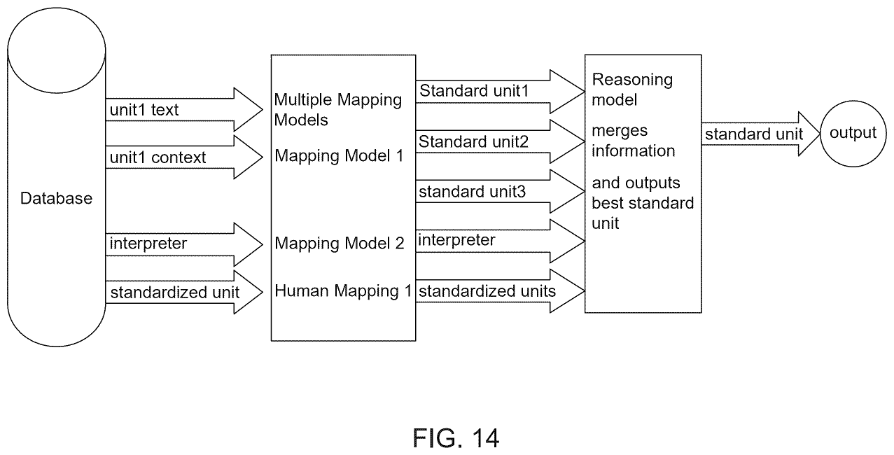

FIG. 14 illustrates a schematic diagram of an exemplary system employing multiple unit mapping models with a reasoning model. In some implementations, multiple unit mapping models may be employed with a reasoning model that selects the most appropriate conversion path whereby the system merges information and outputs best standard unit.

The reasoning model component evaluates outputs from multiple mapping models and selects the most appropriate result. This allows the incorporation of different types of models, such as rule-based systems alongside neural networks while also enabling the system to leverage specialized models trained for specific domains or unit types. In return it provides a mechanism for incorporating human expertise through human-mapped units and improves the overall system accuracy by combining strengths of different approaches.

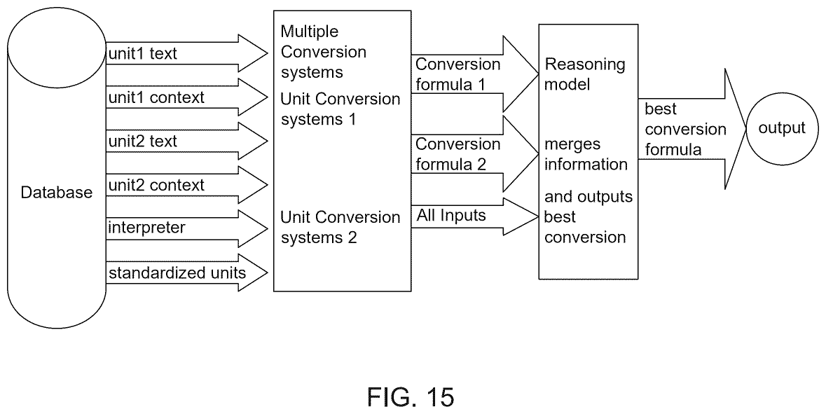

FIG. 15 illustrates a schematic diagram of an exemplary system employing multiple conversion systems with a reasoning model. In some implementations, conversion systems may be employed with a reasoning model that selects the most appropriate conversion path whereby the system merges information and outputs best conversion system.

In the diagram, conversions may come from tables, human inputs, and some from AI models such as transformers. The reasoning system may provide information on why it selected the best conversion or best unit according to how it was trained. To do this, the reasoning model may employ Large Language Models (LLMs) that can provide explanatory capabilities about why a particular standardization was chosen. The reasoning model may employ Retrieval-Augmented Generation (RAG) techniques that access additional reference information to improve decision quality. The reasoning model may employ Cache-Augmented Generation (CAG) that leverages previous standardization decisions to improve consistency.

The reasoning model may employ voting mechanisms that select the most common output across multiple models or confidence-weighted selection that favors outputs from models expressing higher confidence.

Human mapping may be incorporated into this multi-model system, allowing expert-provided mappings to be considered alongside automated approaches. This is useful for rare or specialized units where AI models may have limited training data. Moreover it is possible that a reasoning model gets outputs from multiple models about the same unit or measure or about the same unit conversion.

### User Interface and System Access

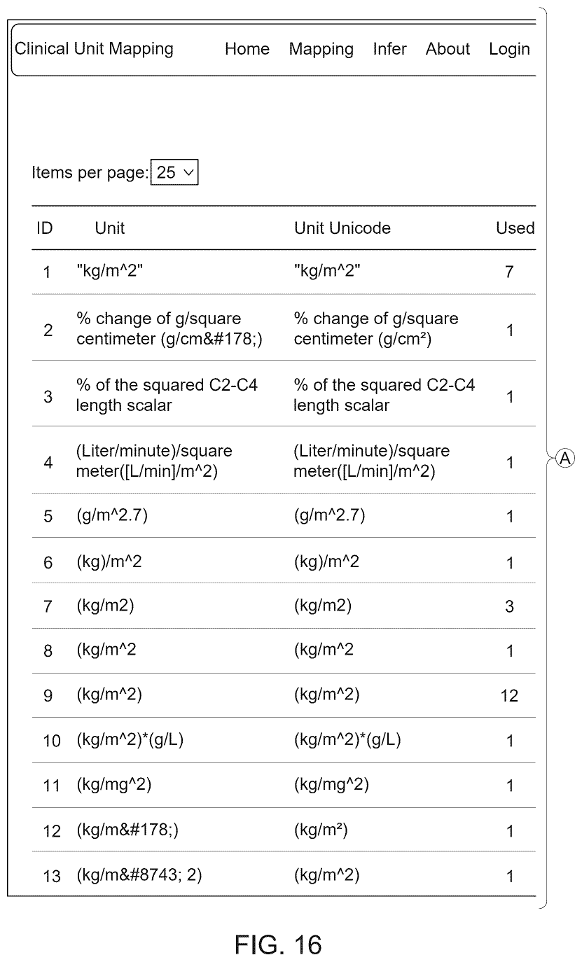

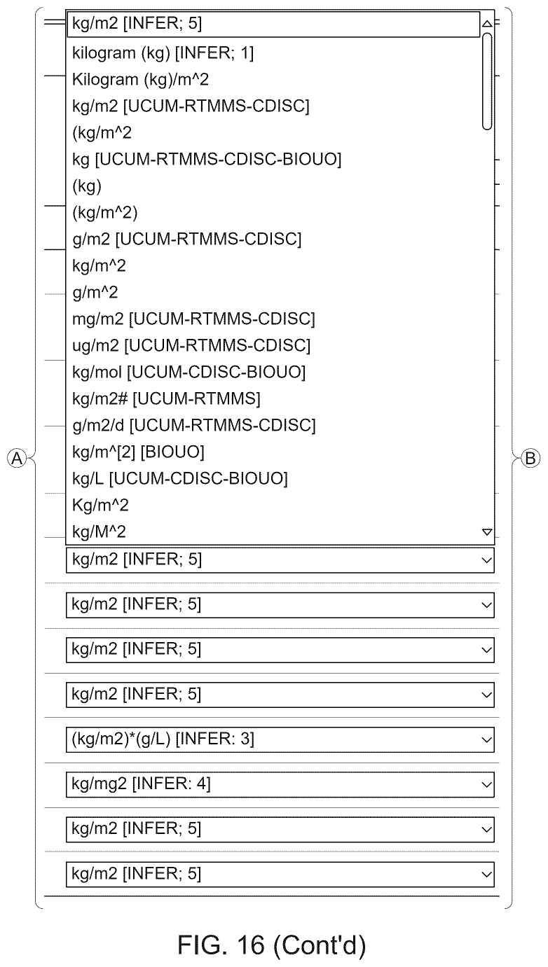

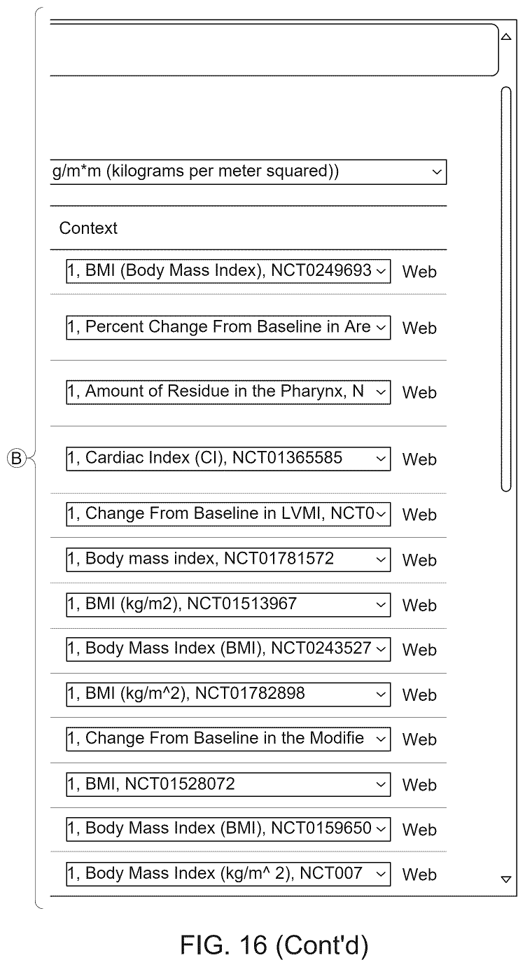

FIG. 16 illustrates a user interface for the AI-assisted unit standardization system. The system may be accessed through various interfaces such as a web-based user interfaces, Application Programming Interfaces (APIs) that enable programmatic access Function calls from integrated software systems, or remote procedure calls across distributed computing environments.

The users may input unit text, unit context, and select an interpreter standard from a dropdown menu. The system processes this information and returns the standardized unit, optionally with conversion capabilities for transforming values between different units.

The system may also provide batch processing capabilities for standardizing large datasets containing multiple units of measure for processing clinical studies data, where numerous measurements with various units may need to be standardized simultaneously.

Integration with Disease Modeling Systems

The unit mapping and conversion systems enhances disease modeling systems by enabling consistent processing of data from disparate sources with heterogeneous unit representations. The unit standardization system ensures that data from different sources is transformed into a uniform representation before being used in modeling. The interpreter-aware standardization enables the same underlying data to be presented according to the preferences of different audiences. The standardization system also facilitates the comparison and combination of such models by converting their inputs and outputs to comparable units.

The systems described may be implemented using various programming languages and frameworks suitable for AI development, such as Python with TensorFlow, PyTorch, or similar machine learning libraries. Deployment options include: Cloud-based services accessible via RESTful APIs On-premises installations for environments with data privacy requirements.

The system may incorporate feedback mechanisms that allow users to report incorrect standardizations, which can be used to continuously improve the AI models through additional training.

The following examples illustrate the operation of the AI-assisted unit standardization system in various scenarios:
Example 1: Standardization with Complete Information Input: Unit text: “kg/m\hat{ }2”; Unit context: “Starting body mass index (starting BMI)”; and Interpreter: “CDISC” Processing. The neural network processes all three inputs. The network suggests “kg/m2” as the standardized form according to CDISC standards. The nearest unit search confirms this is a permitted standardized unit. The output component returns “kg/m2.”
Example 2: Standardization with Partial Information Input: Unit text: “kg/m\hat{ }2”; Unit context: Not provided; Interpreter: “CDISC” Processing. The neural network processes the available inputs. Despite lacking context, the network identifies “kg/m2” as the standardized form for “kg/m\hat{ }2” in CDISC. The nearest unit search confirms this is a permitted standardized unit. The output component returns “kg/m2.”
Example 3: Context-Dependent Standardization Input: Unit text: Not provided; Unit context: “Starting body mass index (starting BMI)”; and Interpreter: “CDISC” Processing. The neural network processes the available inputs. Based on context alone, the network identifies “kg/m2” as the likely unit for BMI in CDISC. The nearest unit search confirms this is a permitted standardized unit. The output component returns “kg/m2.”
Example 4: Standardization with Multi-Model Reasoning Input: Unit text: “mmHg”; Unit context: “Blood pressure measurement”; Interpreter: “CDISC” Processing. Multiple mapping models process the inputs: Model 1 suggests “mmHg” as the standardized form. Model 2 suggests “mm Hg” (with a space) as the standardized form. Human mapping suggests “mm Hg” as the standardized form. The reasoning model evaluates these suggestions, considering that: The CDISC standard prefers “mm Hg” with a space. Two out of three sources agree on “mm Hg.” The reasoning model selects “mm Hg” as the best standardized form. The output component returns “mm Hg”.

### Disease Testing System

To establish and improve modeling system accuracy, a synthetic disease testing framework may be implemented as a comprehensive validation approach. This framework begins with the creation of a synthetic disease model with known behavior, parameters, and progression characteristics, followed by the generation of multiple artificial population datasets that may be based on existing population structures.

The synthetic disease model is then simulated across all populations to establish ground truth disease behavior, providing a benchmark against which model performance can be measured.

A component of this framework is the application of various observer models designed to introduce realistic distortions to the ground truth data. These distortions mimic real-world data collection challenges such as human reporting errors, reporting delays, statistical noise, systematic biases, testing device accuracy limitations, data omissions, and other factors that affect data quality in clinical settings. By introducing these controlled imperfections, the framework creates a more realistic testing environment that better approximates the challenges faced when modeling actual disease outbreaks.

After generating the distorted observations, these data along with the population information are provided as inputs to the disease modeling system. Human modelers may create new models or select existing models from a model library to address the synthetic disease, and the ensemble modeling method previously described is applied to derive an optimized model. The final step involves comparing the optimized model outputs against the original synthetic disease ground truth to quantify modeling accuracy, providing a clear measure of how well the system recovers true disease dynamics from imperfect observations. This synthetic testing framework offers several significant advantages for disease modeling evaluation.

It provides a controlled environment where all underlying disease characteristics are known, enabling precise measurement of modeling system performance. It allows for the calculation of quantifiable metrics for model accuracy under various conditions, helping to identify strengths and weaknesses in current approaches. The framework also yields insights into which base models contribute most effectively to accurate ensemble models, guiding future model development and selection strategies.

Additionally, the framework facilitates the improvement of parameter optimization approaches by allowing systematic testing of different algorithms against known ground truth. It also creates valuable training opportunities for human modelers to recognize effective ensemble construction patterns, accelerating the development of modeling expertise.

The synthetic disease models themselves may be constructed using various methodologies, including deterministic compartmental models, stochastic individual-based models, or hybrid approaches combining elements of both. The essential requirement is that these models produce well-defined disease progression patterns with known parameter values that can serve as the reference standard for validation. This addresses a fundamental challenge in disease modeling validation: for real diseases, the ground truth is always partially unknown, making conclusive validation difficult without a synthetic reference.

The artificial population datasets generated within this framework may represent demographic distributions similar to real populations, such as U.S. states or counties, or may be specifically designed to test modeling challenges like unusual age distributions, geographical variations, or other demographic factors that might influence disease dynamics. This flexibility allows for comprehensive testing across a range of population scenarios, ensuring model robustness across diverse demographic contexts.

Observer models may be carefully designed to replicate the imperfections in real-world disease surveillance systems. These may incorporate time-varying delays in reporting that fluctuate based on system load or resource availability, day-of-week effects that influence reporting patterns (such as reduced weekend reporting), varying detection rates across different demographic groups reflecting healthcare access disparities, and testing capacity limitations that result in undercounting during surge periods. They may also model false positive and false negative rates based on specific testing technology characteristics and missing data patterns that correspond to real-world data collection systems.

By systematically varying the parameters of these observer models, it becomes possible to evaluate the robustness of the disease modeling system under different observation conditions and identify thresholds at which modeling performance degrades significantly. This sensitivity analysis provides valuable insights into which types of data quality issues most severely impact modeling accuracy, helping to prioritize data improvement efforts in real-world applications.

### Comprehensive Population Data Integration

### Data Source Integration

The disease modeling system described herein can utilize diverse population data types, significantly expanding the information available for model development and validation. This integration capability encompasses individual-level data sources such as Electronic Health Records (EHR), Electronic Medical Records (EMR), government database records, longitudinal patient data, and clinical trial participant records. These individual-level sources provide detailed information about specific patients, including demographic characteristics, medical histories, treatment responses, and disease progression trajectories.

In addition to individual-level data, the system can incorporate summary population data from sources such as published clinical trial reports, ClinicalTrials.gov statistical information, epidemiological reports, and public health statistics. These summary sources typically provide statistical distributions, means, medians, and other aggregate measures that characterize populations without disclosing individual-level details.

The system can also implement combined data approaches, including summary statistics derived from individual records, individual records augmented with summary information, and hybrid datasets created through integrating multiple data sources.

The virtual population generation capabilities of the system can merge data from these diverse sources while maintaining statistical consistency with observed populations. This integration allows for the creation of more comprehensive population models that leverage the strengths of both individual-level detail and population-level statistical robustness. When integrating individual-level data, the system can incorporate longitudinal information about each individual, capturing the progression of health states over time. This temporal dimension enriches the modeling capabilities, allowing for more accurate representation of disease progression trajectories and treatment responses over extended periods.

For scenarios where only summary population data is available, the system can generate virtual populations that conform to the statistical constraints while incorporating realistic individual-level variations and correlations. This approach preserves the statistical characteristics of the original population while providing the individual-level granularity needed for microsimulation modeling. The system can also handle mixed scenarios where some characteristics are known at the individual level while others are only available as summary statistics, creating comprehensive virtual populations that make optimal use of all available information.

### Advanced Population Augmentation

When populations have missing data characteristics, the system employs advanced augmentation techniques to fill these gaps while maintaining overall population validity. It can incorporate summary statistics from external sources, applying this information to generate the missing characteristics in a manner consistent with both the external source and the existing population data. The system can also apply demographic data from census or other population-level sources to ensure that the augmented population reflects realistic demographic distributions and correlations.

Environmental data such as weather patterns, geographic information, or socioeconomic indicators can be integrated to provide contextual factors that may influence disease dynamics or treatment responses. When necessary, the system can supplement populations with simulated data that maintains statistical consistency with known parameters, generating synthetic individuals or characteristics that preserve the statistical properties of the reference populations.

This approach provides substantial advantages over traditional imputation methods, which often rely on simple substitution of missing values with means, medians, or regression-based estimates. By preserving the statistical properties of the original data, including distributions, correlations, and outlier patterns, the system creates more realistic augmented populations that better reflect real-world variability. The approach incorporates domain knowledge through rules and objectives, allowing for the implementation of known biological constraints or clinical relationships that might not be captured in statistical patterns alone.

The population augmentation process can also resolve conflicts between data sources with incompatible or contradictory information. By applying prioritization rules and optimization approaches, the system can generate populations that best satisfy the constraints and objectives specified across multiple data sources, creating consensus populations that reflect the most reliable aspects of each contributing source.

To illustrate this capability, consider a case where an HMO has EMR data about a population but lacks information on housing conditions or family size that might influence disease transmission or treatment adherence. This missing information can be augmented using census data or other demographic sources, creating a more comprehensive population model that retains the EMR data's individual-level details while incorporating the additional characteristics from summary sources. The augmented population enables more nuanced modeling of the factors influencing health outcomes while maintaining consistency with both the original EMR data and the supplementary census information.

### Cross-Institutional Modeling

The system facilitates cross-institutional modeling without sharing protected data, addressing a significant challenge in collaborative health research. It accomplishes this by executing models separately on protected populations within each institution's secure environment, combining only the model outputs rather than sharing the underlying data. This approach allows for optimization of ensemble parameters using aggregated model performance metrics while preserving the privacy and security of the original patient data.

Through this federated approach, the system enables the creation of globally applicable models that incorporate international variations in disease presentation, treatment approaches, and healthcare systems. The methodology ensures compliance with regional data regulations such as HIPAA in the United States and GDPR in Europe while still allowing comprehensive disease modeling across institutional and national boundaries.

When multiple institutions each hold datasets with privacy restrictions, traditional modeling approaches would require either sharing the protected data (often not possible due to regulations) or developing separate models that fail to capitalize on the combined knowledge across institutions. The approach described herein allows each institution to execute model components locally on their protected data, sharing only the model, model outputs, or performance metrics which typically don't contain protected information.

This federated approach to model development enables the creation of globally applicable models that incorporate variations across different countries, healthcare systems, and populations. For example, a global diabetes model could incorporate data from multiple countries with different demographic profiles, healthcare access patterns, and treatment approaches, while respecting the data sovereignty requirements of each contributing nation.

### Expanded Model Typologies

### Diverse Model Implementations

The term “model” as used herein encompasses a broad range of implementations, significantly expanding the types of computational approaches that can be incorporated into the disease modeling system. This includes human-provided assumptions and expert rules that capture clinical knowledge and heuristics developed through professional experience. Lookup tables and reference data that provide standardized values or classification criteria based on established medical references can also be integrated as models within the system.

Mathematical equations and statistical formulations, from simple linear relationships to complex differential equations describing disease dynamics, may be supported as model components. Computer programs and algorithms implemented in any programming language can be incorporated, allowing for the integration of specialized computational approaches developed across various research communities and technology platforms.

The system supports unsupervised machine learning models, including clustering algorithms that identify natural groupings in patient data, nearest neighbor methods that predict outcomes based on similarity to known cases, dimensionality reduction techniques that extract key features from complex datasets, and anomaly detection systems that identify unusual disease presentations or treatment responses.

Supervised machine learning models are also supported, including neural networks with various architectures tailored to different data types and prediction tasks. These may include Convolutional Neural Networks (CNN) for image analysis, Recurrent Neural Networks (RNN) and Long Short-Term Memory networks (LSTM) for temporal sequence analysis, and attention mechanisms and transformers for processing complex relationships across variables. Large Language Models (LLM) can be incorporated to extract insights from textual clinical data, while multimodal models capable of processing diverse data types simultaneously can integrate information across different medical measurement modalities.

This broad definition allows the ensemble approach to incorporate traditional statistical models alongside modern machine learning approaches, leveraging the strengths of each methodology to improve overall predictive accuracy. Human-provided expert rules and assumptions remain valuable components of the ensemble, particularly for rare conditions or special cases where statistical data may be limited. By incorporating these expert-derived models alongside data-driven approaches, the system can address edge cases or apply domain-specific knowledge that might not emerge from data analysis alone.

Contemporary machine learning approaches, including deep learning methodologies, may capture complex non-linear relationships in health data that may be missed by traditional statistical methods. The inclusion of these model types extends the system's capability to identify subtle patterns and interactions across multiple variables, potentially revealing previously unrecognized disease mechanisms or risk factors.

### Advanced Input Processing

The expanded model typologies enable processing of complex clinical data types that contain valuable information about disease processes but have traditionally been difficult to incorporate into computational models. Textual clinical notes and reports often contain nuanced descriptions of symptoms, treatment responses, and disease progression that aren't captured in structured data fields. By incorporating natural language processing capabilities, the system can extract relevant features from these unstructured texts and integrate them into the disease modeling process.

Medical imaging data, including X-rays, CT scans, MRI studies, and ultrasound recordings, provide direct visualization of disease manifestations and anatomical changes. Through computer vision and image analysis models, the system can identify relevant features in these images and incorporate them as inputs to the disease modeling process. Time-series data from monitoring devices offer high-resolution temporal information about physiological parameters, which can be analyzed using sequence models to detect patterns, trends, and anomalies relevant to disease progression.

Genomic and other-omics data provides information about the molecular underpinnings of disease processes and treatment responses. By incorporating models capable of analyzing these high-dimensional datasets, the system can integrate genetic and molecular factors into disease predictions. The system can also process both structured and unstructured electronic health record content, extracting relevant clinical information regardless of how it is stored or formatted in the original records.

Model outputs within this expanded typology may represent a diverse range of prediction types. Binary outcomes classify cases into discrete categories such as disease/no disease or treatment success/failure. Categorical classifications assign cases to multiple possible categories, such as disease subtypes or stages. Continuous values predict quantitative measures such as biomarker levels, survival times, or functional assessment scores.

Probability distributions provide not just predicted values but also uncertainty estimates, giving a more complete picture of possible outcomes and their likelihoods. Temporal progressions model how disease states change over time, capturing the dynamic nature of many health conditions. Multivariate predictions simultaneously address multiple outcome variables, acknowledging the complex interconnected nature of health states.

These expanded capabilities maintain compatibility with the ensemble approach previously described, as the system focuses on model outputs and their contributions to overall forecast accuracy rather than internal model structures. The diverse output types can be normalized or transformed as needed to enable direct comparison and integration across different model types. This allows models with fundamentally different internal representations to contribute meaningfully to the ensemble's predictions.

In cases where models produce probability distributions rather than point estimates, the ensemble can incorporate these distributional outputs to better represent uncertainty in predictions. This probabilistic approach provides more informative results than deterministic point estimates alone, particularly for complex disease processes with inherent variability. The ability to handle temporal progressions allows the system to model disease trajectories over time, capturing how conditions evolve, respond to interventions, or interact with comorbidities.

Multi-scale models that operate at different biological levels from cellular to population can be incorporated into the ensemble, allowing the system to connect microscopic disease mechanisms with macroscopic population outcomes. This integration of scales provides a more comprehensive understanding of disease processes and their manifestations across different levels of biological organization.

By expanding the definition of models and the types of data they can process, the system develops more comprehensive and accurate representations of disease processes, leading to improved predictive performance and greater utility for healthcare planning, clinical decision support, and public health interventions. The flexibility to incorporate diverse model types ensures that the system can adapt to new methodological developments and data sources as they emerge, maintaining relevance as medical knowledge and computational techniques continue to advance.

What is claimed is:

1. A system for standardizing units of measure, comprising:
a database comprising standardized units, unit text variations, unit context information, and interpreter standards;
a neural network configured to process at least one of unit text input, unit context input, and interpreter input to generate suggested standardized unit mappings;
and an output component configured to provide a standardized unit based on matched neural network outputs.

2. The system of claim 1, wherein the neural network is a transformer neural network trained on datasets containing unit text, context, the interpreter standards, and correct mapping outcomes.

3. The system of claim 1, further comprising a nearest unit search component configured to match neural network outputs to permitted standardized units from the database; wherein at least one of the unit text input, the unit context input, and the interpreter input is optional, and the neural network is configured to generate the suggested standardized unit mappings based on whichever inputs are provided.

4. The system of claim 1, wherein the interpreter input defines a standard to which a unit should be converted, and wherein same unit text or unit context is mapped to different standardized units based on the interpreter input.

5. The system of claim 1, further comprising a unit conversion system that: receives the standardized unit from the output component; determines conversion formulas between the standardized unit and a target unit; and applies the conversion formulas to produce converted values.

6. The system of claim 5, wherein the unit conversion system comprises multiple unit mapping components that standardize different input units.

7. The system of claim 5, further comprising a reasoning model that selects a most appropriate conversion path from multiple possible conversion paths.

8. A method for validating disease modeling systems using synthetic data, comprising:
creating a synthetic disease model with known behavior parameters and progression characteristics;
generating multiple artificial population datasets based on existing population structures; simulating the synthetic disease model across the artificial population datasets to establish ground truth disease behavior;
applying observer models to introduce realistic distortions to ground truth data, wherein the realistic distortions include at least one of: human reporting errors, delays in reporting, statistical noise, systematic biases, testing device accuracy limitations, and data omissions;
providing distorted observations and the artificial population datasets as inputs to a disease modeling system; generating an ensemble model using the disease modeling system, wherein the ensemble model incorporates multiple individual disease models;
optimizing coefficients of the ensemble model using the distorted observations; and evaluating the disease modeling system by comparing outputs of an optimized ensemble model against the ground truth disease behavior.

9. The method of claim 8, wherein generating the multiple artificial population datasets comprises: selecting one or more reference populations from existing demographic data; and creating variations of the one or more reference populations while maintaining statistical consistency with demographic parameters.

10. The method of claim 8, wherein applying the observer models to introduce the realistic distortions comprises: simulating differential reporting delays for different disease outcomes; applying the statistical noise according to a predetermined distribution; and selectively omitting data according to patterns observed in real-world clinical data collection.

11. The method of claim 8, wherein the ensemble model is optimized using cooperative techniques to determine the coefficients corresponding to individual models in the ensemble model.

12. The method of claim 8, further comprising: identifying which base models from the ensemble model contribute most effectively to matching the ground truth disease behavior; and storing this information to guide model selection for future disease modeling activities.

13. A method for integrating diverse population data sources for disease modeling, comprising:
obtaining individual-level data from at least one of: electronic health records, electronic medical records, government databases, and clinical trial participant records;
obtaining summary population data from at least one of: published clinical trial reports, clinical trials databases, epidemiological reports, and public health statistics;
generating a virtual population that incorporates characteristics from both the individual-level data and the summary population data while maintaining statistical consistency with observed populations;
simulating progression of a biological condition using the virtual population and a plurality of disease models; and
evaluating an aggregate model based on simulation results and observed outcomes from clinical studies.

14. The method of claim 13 wherein generating the virtual population comprises: creating population objects that inherit characteristics from multiple source populations; resolving conflicts between inherited characteristics according to predetermined prioritization rules; and optimizing the virtual population to satisfy statistical objectives derived from the source populations.

15. The method of claim 13, further comprising: identifying populations with missing data characteristics; and augmenting the populations by: incorporating summary statistics from external sources; applying demographic data from census or other population-level sources; integrating environmental data including geographic or socioeconomic information; and supplementing with simulated data that maintains the statistical consistency with known parameters.

16. The method of claim 13, wherein the simulating progression of the biological condition comprises: executing models separately on protected populations at each institution; combining model outputs rather than sharing underlying data; optimizing ensemble parameters using aggregated model performance metrics; and creating globally applicable models that incorporate international variations while maintaining compliance with regional data privacy regulations.

17. The method of claim 13, further comprising: determining that a characteristic is missing from the individual-level data; identifying a corresponding characteristic in the summary population data; and generating the missing characteristic in the virtual population based on the corresponding characteristic from the summary population data.

18. A method for integrating diverse model types in disease progression modeling, comprising:
identifying a plurality of models that predict a progression of a biological condition, wherein the plurality of models includes at least two different model types selected from: human-provided assumptions and expert rules; lookup tables and reference data; mathematical equations and statistical formulations; computer programs and algorithms implemented in a programming language; unsupervised machine learning models; and supervised machine learning models;
processing input data for the plurality of models, wherein the input data includes at least one of: textual clinical notes and reports; medical imaging data; time-series data from monitoring devices; genomic data; and structured and unstructured electronic health record content;
generating an aggregate model that indicates an individual contribution of each model of the plurality of models;
determining the individual contributions of the models with respect to a virtual population; and
evaluating the aggregate model by comparing results with observed outcomes from clinical studies.

19. The method of claim 18, wherein the unsupervised machine learning models include at least one of: clustering algorithms; nearest neighbor methods; dimensionality reduction techniques; and anomaly detection systems.

20. The method of claim 18, wherein the supervised machine learning models include at least one of: neural networks with various architectures; convolutional neural networks; recurrent neural networks; long short-term memory networks; attention mechanisms; transformer models; and large language models.
